(19) United States
(12) Patent Application Publication
(10) Pub. No.: US 2023/0134350 A1
(43) Pub. Date: May 4, 2023

(54) ORGANIC COMPOUND AND ORGANIC ELECTROLUMINESCENCE DEVICE USING THE SAME

(71) Applicants: FENG-WEN YEN, HSIN-CHU (TW); CHIN-MIN TENG, HSIN-CHU (TW)

(72) Inventors: FENG-WEN YEN, TAIPEI (TW); CHIN-MIN TENG, MIAOLI (TW)

(73) Assignee: LUMINESCENCE TECHNOLOGY CORP., HSIN-CHU (TW)

(21) Appl. No.: 17/449,258

(22) Filed: Sep. 28, 2021

(51) Int. Cl. C07D 519/00; H01L 51/00

(52) U.S. Cl. CPC C07D 519/00; H01L 51/0072; H01L 51/0067; H01L 51/0074; H01L 51/0094; H01L 51/0073; H01L 51/0056; H01L 51/5016

(57) ABSTRACT

An organic compound having the following formula (I) is described:

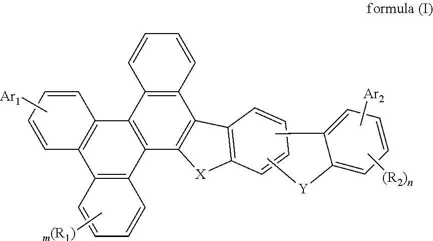

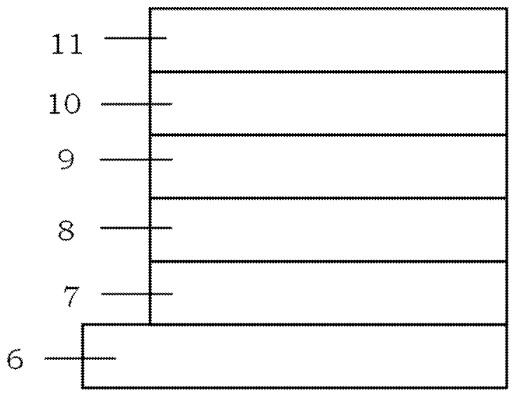

The application of the organic compound in an organic electroluminescence device is also described.

### BACKGROUND

### Field of Invention

The present invention relates generally to a compound, and, more specifically, to an organic electroluminescence device.

### Description of Related Art

An organic electroluminescence (organic EL) device, i.e., organic light emitting diodes (OLEDs) that make use of organic compounds, are becoming increasingly desirable than before. One of the organic compounds has the following formula:

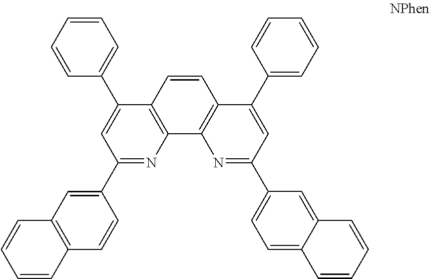 [SMILES: c1ccc(-c2cc(-c3ccc4ccccc4c3)nc3c2ccc2c(-c4ccccc4)cc(-c4ccc5ccccc5c4)nc23)cc1]

An organic EL device is a light emitting diode (LED) in which the light emitting layer is a film made from organic compounds, which emits light in response to an electric current. The light emitting layer containing the organic compound is sandwiched between two electrodes. The organic EL device is applied to flat panel displays due to its high illumination, low weight, ultra-thin profile, self-illumination without back light, low power consumption, wide viewing angle, high contrast, simple fabrication methods and rapid response time.

However, there is still a need for improvement in the case of use of those organic materials in an organic EL device of some prior art displays, for example, in relation to the half-life, current efficiency or driving voltage of the organic EL device.

As used herein, “top” means furthest away from the substrate, while “bottom” means closest to the substrate. Where a second layer is described as formed on or onto a first layer, the second layer is formed further away from substrate. There may be other layers between the second layer and the first layer, unless it is specified that the second layer is “in contact with” the first layer. For example, a cathode may be described as formed onto an anode, even though there are various organic layers in between.

### SUMMARY

An aspect of the present disclosure provides an organic compound is represented by the following formula (I):

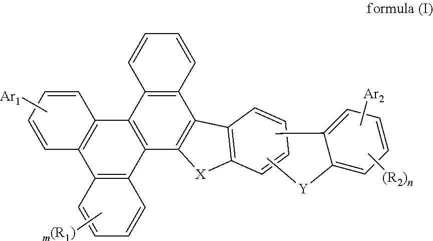

wherein m is an integer of 0 to 11;
wherein n is an integer of 0 to 3;
wherein X is O, S, C(R₃)(R₄), N(Ar₃), or Si(R₅)(R₆);
wherein Y is O, S, C(R₇)(R₈), N(Ar₄), or Si(R₉)(R₁₀);
wherein Ar₁ and Ar₂ are each independently a hydrogen atom, a halide, a substituted or unsubstituted arylamine group having 5 to 50 ring atoms, a substituted or unsubstituted heteroarylamine group having 5 to 50 ring atoms, a substituted or unsubstituted aryl group having 5 to 50 ring atoms, or a substituted or unsubstituted heteroaryl group having 5 to 50 ring atoms; wherein Ar₃ and Ar₄ are each independently a hydrogen atom, a substituted or unsubstituted aryl group having 5 to 50 ring atoms, or a substituted or unsubstituted heteroaryl group having 5 to 50 ring atoms; and wherein R₁ to R₁₀ are independently selected from the group consisting of a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, and a substituted or unsubstituted heteroaryl group having 3 to 30 carbon atoms.

Another aspect of the present disclosure provides an organic compound represented by the following formula (I):

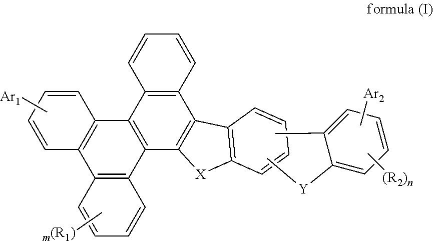

wherein X and Y are independently O, S, NR, C(CH₃)₂, C(CH₂CH₃)₂, Si(CH₃)₂ or Si(CH₂CH₃)₂;
wherein R is selected from the group consisting of aryl, aza-aryl, and combinations thereof;
wherein R is optionally substituted with phenyl, biphenyl, tricyclic aryl or tricyclic aza-aryl;
wherein Ar₁ and Ar₂ are independently absent or selected from the group consisting of methyl, isopropyl, isobutyl, phenyl, pyridinyl, and combinations thereof;
wherein m is an integer of 0, 1, 2, 3, 4, 5, 6, 7, 8, 9, 10 or 11; and
wherein n is an integer of 0, 1, 2 or 3.

The application of the organic compound in an organic electroluminescence device is also described. The electroluminescence device, also a red, amber or yellow electroluminescence device, may serve as a tail light or a turn light of a car having longer half-life, higher current efficiency, higher luminous efficiency and/or lower driving voltage.

### BRIEF DESCRIPTION OF THE DRAWINGS

The FIGURE shows one embodiment of the organic EL device of the present invention. In the organic EL device, a hole injection layer 7 is deposited onto a transparent electrode layer 6 (e.g., ITO). A hole transport layer 8 is deposited onto the hole injection layer 7. A fluorescence or phosphorescence emitting layer 9 is deposited onto the hole transport layer 8. An electron transport layer 10 is deposited onto the emitting layer 9. A metal electrode 11 is deposited onto the electron injection layer 10.

### DETAILED DESCRIPTION

Plural embodiments of the present disclosure are disclosed through drawings. For the purpose of clear illustration, many practical details will be illustrated along with the description below. It should be understood that, however, these practical details should not limit the present disclosure. In other words, in embodiments of the present disclosure, these practical details are not necessary. In addition, for the purpose of simplifying the drawings, some conventional structures and components are simply and schematically depicted in the figures.

Aiming at a gap or a specific layer calibrated by the receptor component, to research and develop new organic compounds having functions of OLED photoelectric mechanism and actually solving technical problems, is not only one of the main ideas and approaches in the research and development, but also key technical basis for filing patent applications. The above-mentioned research methods of photoelectric mechanism have become the key work for the research and development of organic compounds in this field. Therefore, it is not suitable to deviate from the research methods in this field when determining the technical problems actually solved by the present disclosure. When the concept of the present disclosure is analyzed, it will help to more accurately determine the technical problems actually solved by the compound invention, and to lay a solid foundation for the judgment of technical enlightenment, if the above-mentioned research methods of those skilled in the art is followed, if the process of inventing an organic compound and an OLED using the same is experienced, and if the applicant's technical contributions are essentially grasped.

It is to be understood that although particular phrases used herein, such as “first”, “second”, “third”, and so on, are used to describe different components, members, regions, layers, and/or sections, these components, members, regions, layers, and/or sections should not be limited by these terms. These phrases are used to distinguish one component, member, region, layer, or section from another component, member, region, layer, or section. In this way, a first component, member, region, layer, and/or section to be described below may be referred to as a second component, member, region, layer, and/or section, without departing from the spirit and scope of the present disclosure.

Spatially relative phrases, such as “onto”, “on”, “under”, “below”, “underlying”, “beneath”, “above”, and so on used herein, are used for facilitating description of a relation between one component or feature and another component or feature depicted in the drawings. Therefore, it can be understood that, in addition to directions depicted in the drawings, the spatially relative terms mean to include all different orientations during usage or operations of the device. For example, it is assumed that a device in a figure is reversed upside down, a component described as being “under”, “below”, or “beneath” another component or feature is oriented “onto” or “on” the other component or feature. Therefore, these exemplary terms “under” and “below” may include orientations above and below. The device may be otherwise oriented (e.g., turned by 90 degrees, or other orientations), and the spatially relative terms used herein should be explained accordingly.

Accordingly, it may be understood that when a component or a layer is referred to as being “onto”, “on”, “connected to”, or “coupled to” another component or another layer, it may be immediately on the other component or layer, or connected to or coupled to the other component or layer, or there may be one or more intermediate components or intermediate layers. Further, it can be understood that when a component or a layer is referred to as being “between” two components or two layers, it may be the only component or layer between the two components or layers, or there may be one or more intermediate components or intermediate layers.

Terminologies used herein are only for the purpose of describing particular embodiments, but not limiting the present disclosure. The singular form of “a” and “the” used herein may also include the plural form, unless otherwise indicated in the context. Accordingly, it can be understood that when there terms “include” or “comprise” are used in the specification, it clearly illustrates the existence of a specified feature, bulk, step, operation, component, and/or member, while not excluding the existence or addition of one or more features, bulks, steps, operations, components, members and/or groups thereof. “And/or” used herein includes any and all combinations of one or more related terms that are listed. When a leading word, such as “at least one of”, is added ahead of a component list, it is to describe the entire component list, but not individual components among the list.

It is to be understood that when a molecular fragment is described as being a substituent or otherwise attached to another moiety, its name may be written as if it were a fragment (e.g., aryl, phenyl, biphenyl, dibenzofuranyl, naphthyl) or as if it were the whole molecule (e.g., arylene, benzene, phenylbenzene, dibenzofuran, naphthalene). As used herein, these different ways of designating a substituent or attached fragment are considered to be equivalent.

The terms “substituted” and “substitution” refer to a substituent bonded to the relevant position, e.g., a carbon or nitrogen. Accordingly, for example, when R₁ represents mono substitution, one R₁ must not be H. Similarly, when R₁ represents at least di substitutions, at least two R₁ must not be H. In addition, when R₁ represents no substitution, R₁, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine.

As used herein, a maximum possible number of substitutions in a ring structure will depend on the total number of available valencies in the ring atoms. See

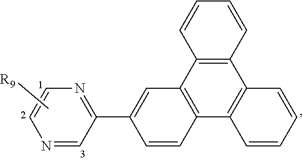

for example, there are three available valencies in the ring atoms of the left heteroaryl ring, so the maximum possible number of substitutions of R₉ substitution is 3.

As used herein, when a single ring of a compound's formula is substituted a non-symmetrical polycyclic aryl, the formula may represent at least two compounds. For one example, the formula

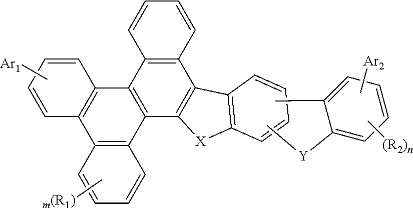

represents a first formula of

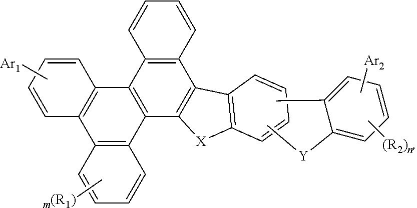

and represents a second formula of

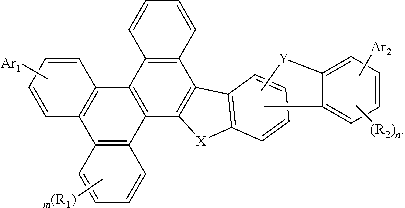

Even if the scope of the patent application only draws the first formula, it is not limited to it. That is, the formula of the first formula is to further comprise the second formula. See the following for reference.

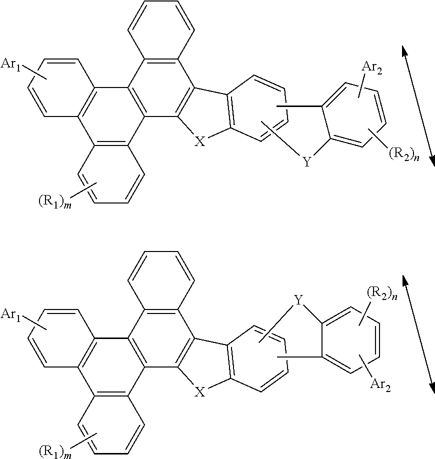

As used herein, terms “R₁”, “R₂”, “R₃”, “R₄”, “R₅”, “R₆” . . . or “R₁₄” may independently be hydrogen or a substituent selected from the group consisting of aryl, alkyl, alkylphenyl, halogen, phenyl, methylphenyl, pyridyl, biphenyl, pyridylphenyl, 9,9-dialkylfluorenyl, dibenzofuranyl, benzothienyl, carbazolyl, methyl, butyl, n-butyl, hexyl, propyl, isopropyl, hexylphenyl, triazinyl, diazinyl, naphthyl, heteroaryl, aralkyl , Trifluoromethyl, cyano, nitro, trimethylsilyl, silyl, aryl substituted or unsubstituted by methyl or hexyl, biphenyl, pyridylphenyl, m-terphenyl, diiso Butylcarbazolyl, phenylcarbazolyl, dimethylcarbazolyl, cyano, phenyl, dicyanophenyl, nitro, cycloalkyl, heterocycloalkyl, aromatic group, aromatic amine group, heteroarylamine group, deuterium, alkoxy, amino, silyl, fluorenyl, benzofluorenyl, anthracenyl, phenanthryl, pyrenyl, alkenyl, cycloalkenyl, heteroalkenyl, alkynyl, carbonyl , Carboxylic acid, ether, ester, glycol, isonitrile, thio, sulfinamide, sulfonyl, phosphoric acid, triphenylene, benzimidazole, dicarbazolyl, diphenylphosphine oxide, phenanthroline group, dihydroacridinyl, phenothiazinyl, phenoxazinyl, dihydrophenazinyl, diphenylamino, triphenylamino, phenyldibenzofuranylamino, phenyldiphenyl thioanilino, and combinations thereof. In addition, when each of R₁, R₂, R₃, R₄, R₅, R₆. . . or R₁₄ represents a substituent, two adjacent substituents, for example, two adjacent R substituents may be optionally bonded (connected) or fused together to form a single ring structure (such as benzene), or to form fused rings (also polycyclic aromatic groups such as naphthalene. The fused rings are formed together with the substituted.

As used herein, if the term “a first integer to a second integer” is used to express a plurality of solutions, it may cover the first integer, the second integer, and each integer between the first and the second integers. That is to say, when the term “a first integer to a second integer” expresses a plurality of solutions, all of its integers are parallel technical solutions. In this case, the term “a first integer to a second integer” is not used to express a numerical range. For example, 1 to 4 covers 1, 2, 3, 4 and does not include 1.5. For another example, 0 to 3 cover 0, 1, 2, and 3, wherein 0, 1, 2, and 3 are technical solutions in parallel. For more example, 5 to 50 covers 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, . . . , and 50, among which 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, . . . , and 50 are parallel technical solutions. These solutions may be, for example, the number of ring atoms of arylamine group, heteroarylamine group, aryl group or heteroaryl group, the number of substituents, or the number of carbon atoms. It is noted that “a maximum possible number of substitutions” is also a kind of integer.

As used herein, “combinations thereof” indicates that one or more members of the applicable list are combined to form a known or chemically stable arrangement that one of ordinary skill in the art can envision from the applicable list. For example, two phenyl groups can be combined (bonded) to form a biphenyl group. If a methyl phenyl group substituted for a first C is combined to a methyl group substituted for a second C adjacent to the first C, a naphthalene is formed (together with the adjacent Cs). The monocyclic aromatic group and the polycyclic aromatic group can be bonded (connected) together through a direct bond (single bond), or can be condensed to form two adjoining rings in which two carbons are common the two adjoining rings. Alkyl and deuterium can be combined to form partially or fully deuterated alkyl. Halogen and alkyl can be combined to form haloalkyl. Halogen, alkyl and aryl can be combined to form haloaralkyl.

The following description of various terms related to substituents, one of the intentions, is to show that some substituents are mutually replaceable and/or have common functions. In other words, in the field of the present disclosure, these substituents may be of the same category.

The term “alkyl” refers to and includes both straight and branched chain alkyl radicals. Preferred alkyl groups are those containing 30 or fewer carbon atoms, preferably 1 to 20 carbon atoms, more preferably 1 to 15 carbon atoms, and most preferably 1 to 12 carbon atoms. Suitable alkyl groups include methyl, ethyl, propyl, isopropyl, butyl, n-butyl, hexyl, n-hexyl, 1-methylethyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “aryl” or “aromatic group” as used herein are interchangeable with each other and contemplates monocyclic aromatic groups (or monocyclic aryl), polycyclic aromatic groups (or polycyclic aryl), fused ring hydrocarbon units, and combinations thereof. The polycyclic aryl may have two, three, four, five, or more rings in which two carbons are common to two adjoining rings (meaning that the two adjacent rings are “fused ”). That is, a polycyclic aryl comprises a bicyclic aryl having two rings. A polycyclic aryl also comprises a tricyclic aryl having three rings. In a polycyclic aromatic group, at least one of the polycyclic rings is an aryl, e.g., the other rings can be aryls, heteroaryls, cycloalkyls, cycloalkenyls, and/or heterocycles. In terms of the number of carbon atoms, preferred aromatic groups are those containing 30 or fewer carbon atoms, preferably 6 to 30 carbon atoms, more preferably 6 to 20 carbon atoms, and most preferably 6, 10 or 12 carbon atoms.

Suitable aryl includes but not limited to phenyl, 3-biphenyl, 2-biphenyl, 4-biphenyl, fluorenyl, fluoranthene, benzanthracene, benzo[c]phenanthrene, benzofluorene, 9,9-dialkylfluorenyl, 9,9-dimethylfluorene, naphthalene, phenanthrene, anthracene, triphenylene, pyrene, phenanthrene, perylene, terphenyl, terphenyl, m-terphenyl, p-terphenyl, o-terphenyl, tetraphenyl, phenalene, 9H-fluorene, and azulene. Among the suitable aromatic groups, preferred include phenyl, 3-biphenyl, 2-biphenyl, 4-biphenyl, fluorenyl, fluoranthene, benzanthracene, benzo[c]phenanthrene, benzofluorene, 9,9-dimethylfluorene, naphthalene, phenanthrene, anthracene, triphenylene, pyrene, chrysene, perylene, terphenyl, fluorene and naphthalene. In addition, the above-mentioned “aryl” or “aromatic group” may be optionally substituted, for example, the two hydrogen atoms commonly bonded on the same carbon atom of benzofluorene may be further substituted with two methyl groups, which product may be named dimethyl-benzofluorene. As another example, the phenyl group may be further substituted with methyl, hexyl or pyridyl.

Among the listed aryls, the “aza” designation in the fragments i.e., aza-aryl, aza-dibenzofuran, aza-dibenzothiophene, etc. means that one or more of the C—H groups in the respective fragment can be replaced by a nitrogen atom, for example, and without any limitation, aza-triphenylene encompasses both dibenzo[f,h]quinoxaline and dibenzo[f,h]quinoline. One of ordinary skill in the art can readily envision other nitrogen analogs of the aza-derivatives described above, and all such analogs are intended to be encompassed by the terms as set forth herein.

It is noted that if the compounds drawn in this article, whether in the specification or the scope of the patent application, contain dibenzofuran, the substantial meaning of the drawn dibenzofuran is sufficient to comprise polycyclic heteroaromatic groups containing dibenzothiophene. The O of dibenzofuran and S of dibenzothiophene are elements of the same group. The structure and chemical properties of O and S are very similar, and they may therefore be used instead of each other. For example, even if the scope of the patent application only draws the polycyclic heteroaromatic compound containing dibenzofuran on the left side of the figure, it is not limited to this. In addition, the S of the dibenzothienyl group has to be changed to Se for similar reasons, because Se and S are elements of the same group, with very similar structures and chemical properties, and can be used instead of each other. Moreover, for similar reasons, O, S, C(CH₃)₂, C(CH₂CH₃)₂, Si(CH₃)₂ and Si(CH₂CH₃)₂ are similar structures having similar chemical properties, and therefore can be used instead of each other.

If the C of the dialkyl group of the 9,9-dialkylfluorenyl group is changed to Si, the changed group is not for defining a polycyclic aromatic group, but a polycyclic heteroaromatic group. Broadly speaking, Si and C are elements of the same family, their structure and chemical properties are very similar, and they can therefore be used instead of each other. Accordingly, changing the C of the dialkyl group of the 9,9-dialkylfluorenyl group to Si is a suitable example. In addition, the alkyl group attached to Si can also be changed to Rs, and each Rs can be the same or different alkyl groups or other substituents. That is, each Rs can be H (hydrogen) or a substituent selected from the group consisting of deuterium, halogen, alkyl, cycloalkyl, heteroalkyl, heterocycloalkyl, aralkyl, alkoxy, aryloxy, amino, silyl, alkenyl, cycloalkenyl, heteroalkenyl, alkynyl, aryl, heteroaryl, and combinations thereof. Preferred Rs is selected from the group consisting of alkyl, aryl, tolyl, pyridyl, hexylphenyl, naphthyl, and combinations thereof.

The following substituents are pyridyl and monocyclic aza-phenyls (e.g., pyrimidinyl or triazinyl) containing two or more nitrogen atoms that are not adjacent to each other on the ring:

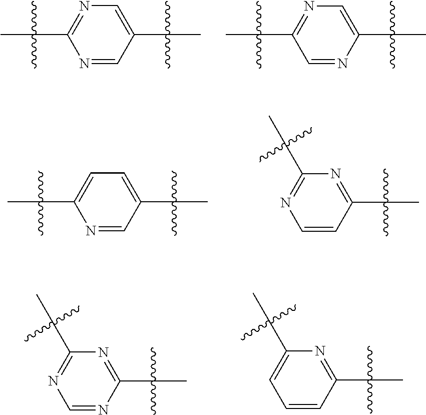 [SMILES: *c1cnc(*)nc1 ; *c1cnc(*)cn1 ; *c1ccc(*)nc1 ; *c1ccnc(*)n1 ; *c1ncnc(*)n1 ; *c1cccc(*)n1]

Likes phenylene, they have three pairs of π electrons that resonate with each other and have similar chemical properties. Therefore, if a compound preferably comprises one of the substituents, other substituents may also be preferred.

As used herein, the terms “heteroaryl” or “heteroaromatic group” are interchangeable with each other. “Heteroaryl” or “heteroaromatic group” may be selected from the group consisting of a monocyclic heteroaromatic group containing one, two, three, four, five or more heteroatoms, a polycyclic heteroaromatic group having two or more rings, and combinations thereof. Heteroatoms include, but are not limited to, O, S, N, P, B, Si and Se. In many cases, O, S, N, Si and Se is the preferred heteroatom. The “monocyclic heteroaromatic group” is preferably a single ring having 5 or 6 ring atoms, and the ring may have one to six heteroatoms. A “polycyclic heteroaromatic group” may have two, three, four, five, six or more rings, where two carbons are common to two adjoining rings (meaning that the rings are “fused”). A polycyclic heteroaromatic group can be named a bicyclic heteroaromatic group if it has two rings; if it has three rings, it can be named a tricyclic heteroaromatic group, and so on. In a polycyclic heteroaromatic group, at least one of the rings is a heteroaryl, e.g., the other rings can be cycloalkyls, cycloalkenyls, aryl, heterocycles, and/or heteroaryls. In terms of the number of carbon atoms, the polycyclic heteroaromatic groups can have from one to six heteroatoms per ring of the polycyclic heteroaromatic groups. Preferred heteroaryl groups are those containing 30 or fewer carbon atoms, preferably 3 to 30 carbon atoms, more preferably 3 to 20 carbon atoms, and most preferably 3 to 12 carbon atoms. Suitable heteroaromatic groups may include dibenzothienyl, dibenzofuranyl, carbazolyl, pyridyl, triazinyl, diazinyl, 1,3,5-triazinyl, quinazoline, quinoxaline, benzoquinazoline , Pyrimidine, dibenzoselenophene, furan, thiophene, benzofuran, benzothiophene, benzoselenophene, indolocarbazole, pyridylindole, pyrrolodipyridine, pyrazole, imidazole, triazole, oxazole, thiazole, oxadiazole, oxtriazole, dioxazole, thiadiazole, pyridazine, pyrazine, oxazine, oxthiazine, oxadiazine, indole, benzimidazole, indazole, inoxa oxazine, benzoxazole, benzisoxazole, benzothiazole, quinoline, isoquinoline, cinnoline, naphthyridine, phthalazine, pteridine, xanthene, acridine, phenazine, phenazine thiazine, phenoxazine, benzofuropyridine, furobipyridine, benzothienopyridine, thienodipyridine, benzoselenophenopyridine and selenophenopyridine, 1,2-azaborane, 1,3-azaborane, 1,4-azaborane, borazine, and other nitrogen analogs of the aza-derivatives described above. Among the suitable heteroaromatic groups, preferred include dibenzothiophene, dibenzofuran, carbazole, pyridine, quinazoline, quinoxaline, benzoquinazoline, dibenzoselenophene, indolocarbazole, imidazole, triazine, benzimidazole, 1,2-azaborane, 1,3-azaborane, 1,4-azaborane, borazine, and other nitrogen analogs of the aza-derivatives described above. In addition, the “heteroaryl” or “heteroaromatic group” may be optionally substituted. For example, the carbazolyl group can be further substituted with two isobutyl groups, which is called diisobutylcarbazole.

Among the aryl and heteroaryl groups listed above, aryl, alkyl, alkylphenyl, halogen, phenyl, methylphenyl, pyridyl, biphenyl, pyridylphenyl, 9 ,9-dialkylfluorenyl, dibenzofuranyl, dibenzothienyl, dibenzoselenophene, carbazolyl, methyl, ethyl, isopropyl, n-butyl, n-hexyl, triazine , Pyrimidine, quinoxaline, quinazoline, benzoquinazoline, pyrene, perylene, naphthalene, anthracene, triphenylene, fluoranthene, dimethyl-benzofluorene, phenanthrene, phenanthrene, benzo[c]phenanthrene, benzanthracene, indolocarbazole, imidazole, pyrazine, and benzimidazole, as well as the aza-derivatives or analogs thereof, are of particular interest.

The terms alkyl, heteroaryl, aryl, and heterocyclic group, as used herein, are independently unsubstituted, or independently substituted, with one or more general substituents selected from the group consisting of methyl, ethyl, propyl, butyl, hexyl, halogen, alkyl, phenyl, methylphenyl, pyridyl, biphenyl, pyridylphenyl, 9,9-dialkylfluorenyl, two benzofuranyl, dibenzothienyl, dibenzoselenophene, amino, silyl, cyano, trifluoromethyl, cyanobenzene, dicyanobenzene, benzofluorene, deuterium, halogen, alkyl , Cycloalkyl, heteroalkyl, aryl, aralkyl, alkoxy, aryloxy, cycloamino, alkenyl, cycloalkenyl, heteroalkenyl, alkynyl, heteroaryl, acyl, carbonyl , Carboxylic acid, ether, ester, nitrile, isonitrile, thio group, sulfinyl group, sulfinyl group, phosphine group, and combinations thereof.

The terms “halogen” and “halide” are used interchangeably and refer to fluorine, chlorine, bromine, or iodine. The term “trifluoromethyl” refers to a —CF₃ radical. The term “cyano” refers to a —C═N radical. The term “nitro” refers to a −NO₂ radical.

As used herein, abbreviations refer to materials and/or films as follows:

LiQ: 8-hydroxyquinolato-lithium
EIL: electron injecting layer
ETL: electron transport layer
ETM: electron transport material
EML: emitting layer
EBL: electron blocking layer
HTL: hole transporting layer
HIL: hole injection layer
ITO: indium tin oxide
EL: electroluminescence
HBL: hole blocking layer
HAT-CN: Dipyrazino [2,34:2 ,3-] quinoxaline-2 ,3 ,6,7,10,11-hexacarbonitrile
NPB: N,N-bis(naphthalene-1-yl)-N,N-bis(phenyl)-benzidine
NPhen: 2,9-bis(naphthalene-2-yl) -4,7-diphenyl-1,10-phenanthroline
BAlq: Bis(2-methyl-8-quinolinolate)-4-(phenylphenolato)aluminium

In the present disclosure, after producing a red OLED, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

In some embodiments, an organic compound is represented by the following formula (I):

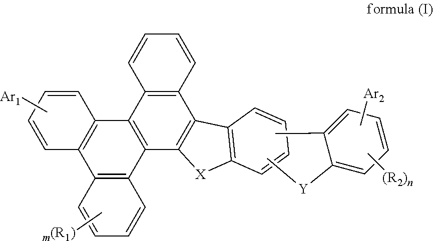

wherein m is an integer of 0 to 11;
wherein n is an integer of 0 to 3;
wherein X is O, S, C(R₃)(R₄), N(Ar₃), or Si(R₅)(R₆);
wherein Y is O, S, C(R₇)(R₈), N(Ar₄), or Si(R₉)(R₁₀);
wherein Ar₁ and Ar₂ are each independently a hydrogen atom, a halide, a substituted or unsubstituted arylamine group having 5 to 50 ring atoms, a substituted or unsubstituted heteroarylamine group having 5 to 50 ring atoms, a substituted or unsubstituted aryl group having 5 to 50 ring atoms, or a substituted or unsubstituted heteroaryl group having 5 to 50 ring atoms;
wherein Ar₃ and Ar₄ are each independently a hydrogen atom, a substituted or unsubstituted aryl group having 5 to 50 ring atoms, or a substituted or unsubstituted heteroaryl group having 5 to 50 ring atoms; and
wherein R₁ to R₁₀ are independently selected from the group consisting of a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, and a substituted or unsubstituted heteroaryl group having 3 to 30 carbon atoms.

In some embodiments, Ar₁ and Ar₂ may be independently selected from the group consisting of the following substituents:

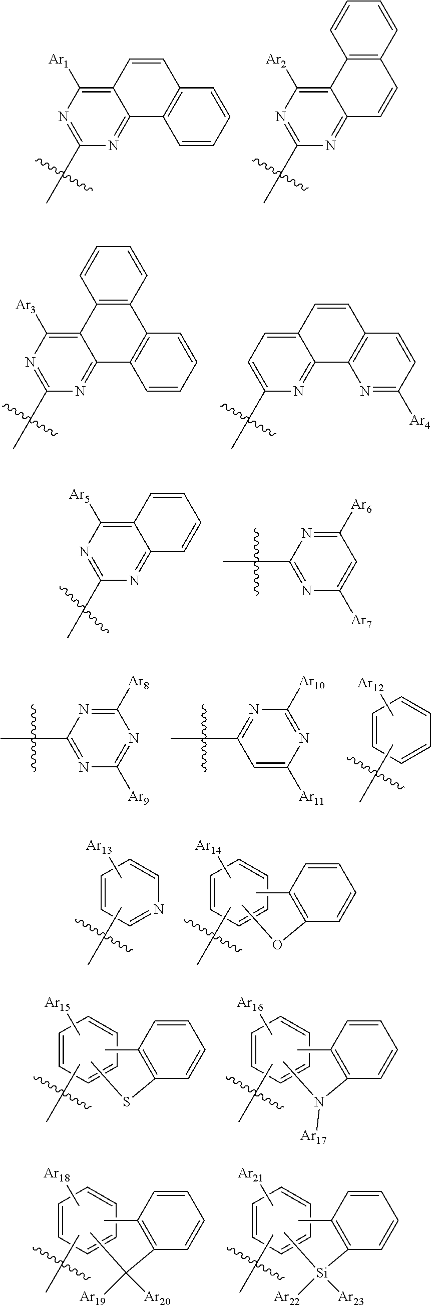

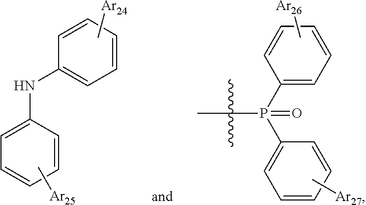

and wherein Ar5˜Ar27 are each independently a hydrogen atom, a substituted or unsubstituted aryl group having 5 to 50 ring atoms, or a substituted or unsubstituted heteroaryl group having 5 to 50 ring atoms.

Ar₁ and Ar₂ may be independently selected from the group consisting of the following substituents:

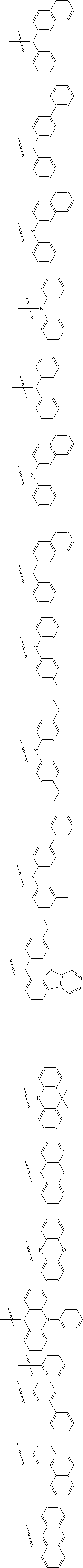

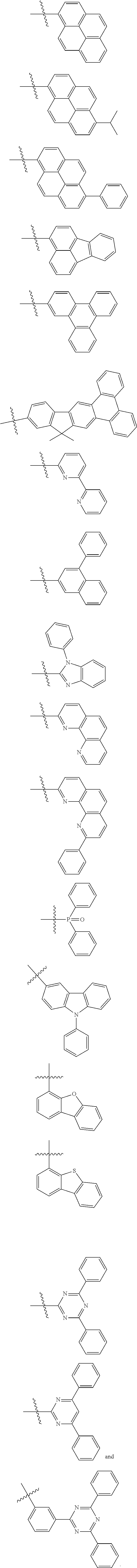

In some embodiments, Ar₃ and Ar₄ may be independently selected from the group consisting of the following substituents:

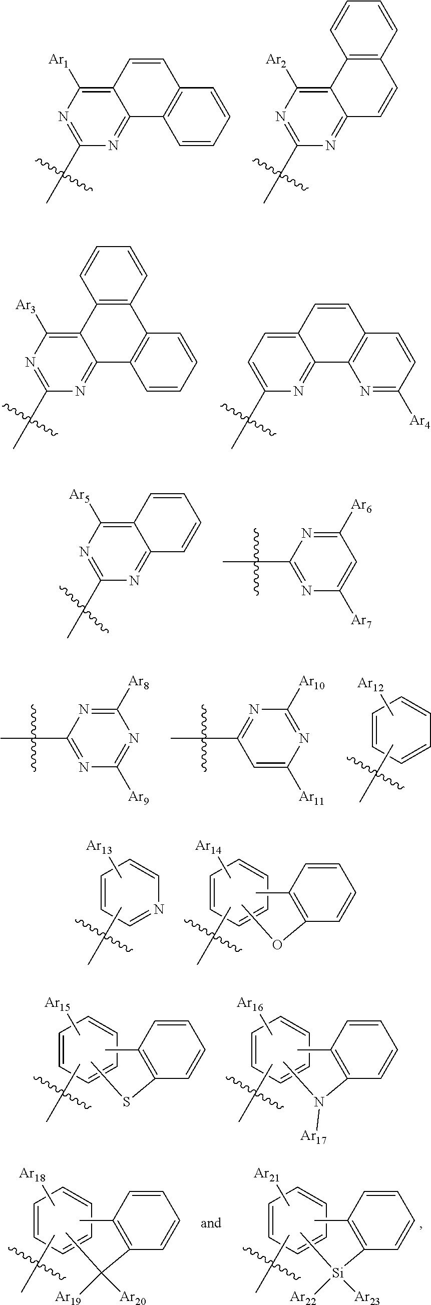

and Ar₅˜Ar₂₃ are each independently a hydrogen atom, a substituted or unsubstituted aryl group having 5 to 50 ring atoms, or a substituted or unsubstituted heteroaryl group having 5 to 50 ring atoms.

Ar₃ and Ar₄ may be independently selected from the group consisting of the following substituents:

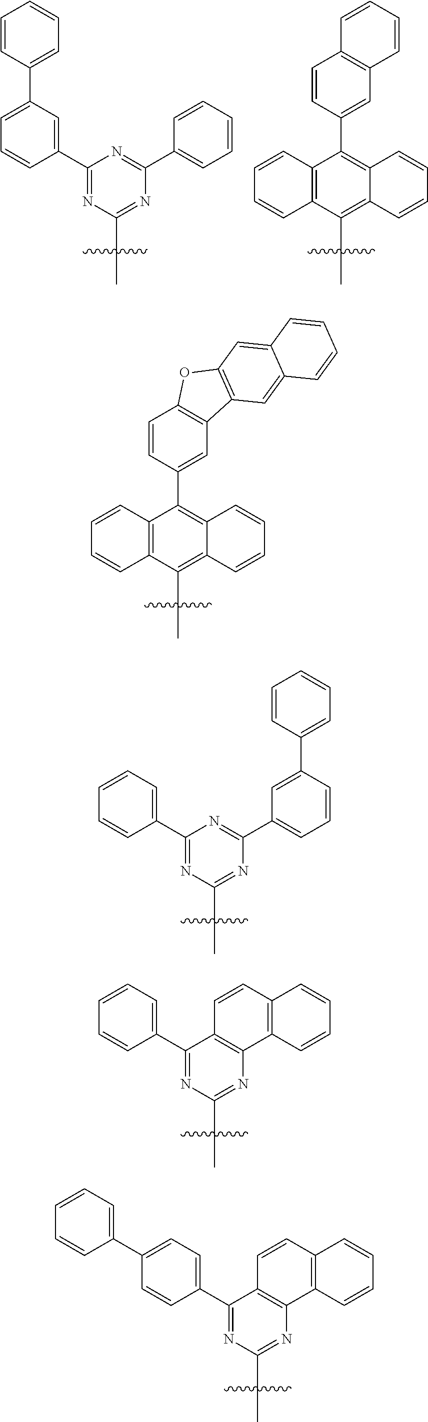

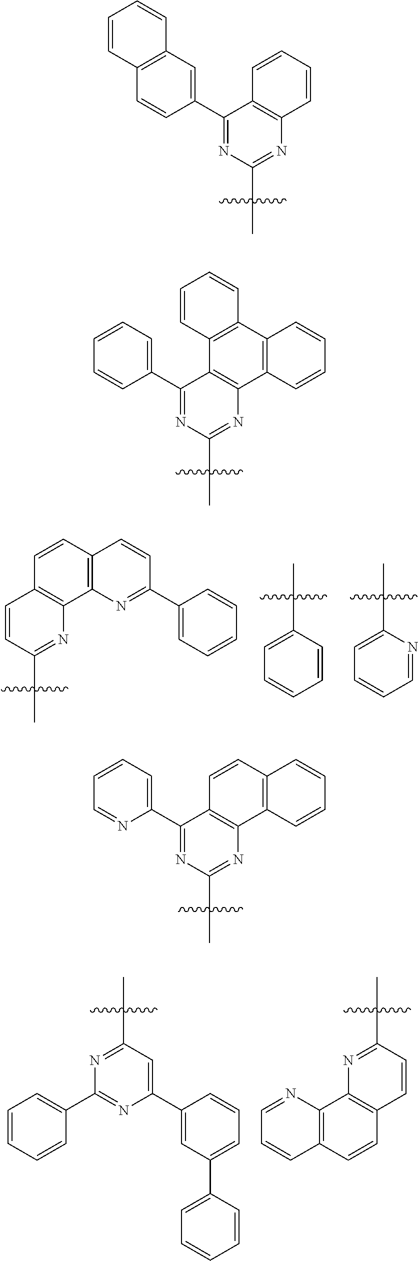

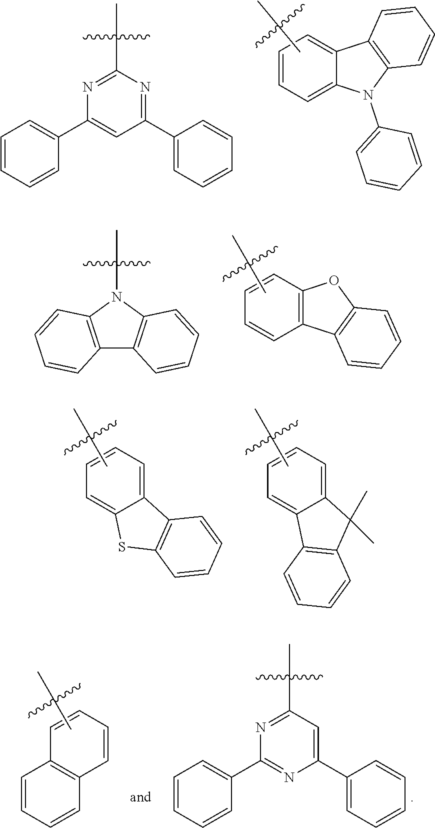

Preferably, the organic compound may be selected from the group consisting of the following compounds:

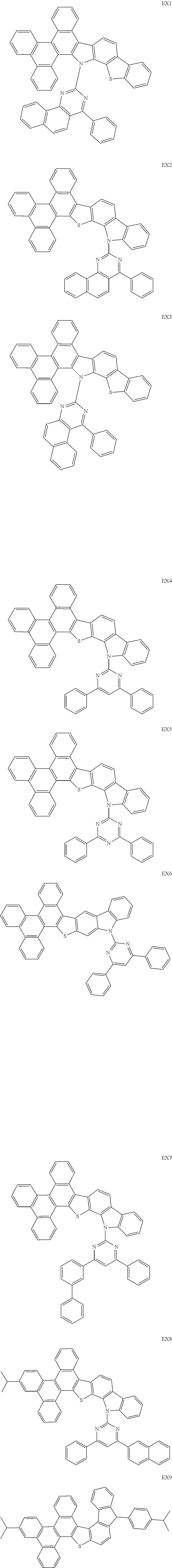

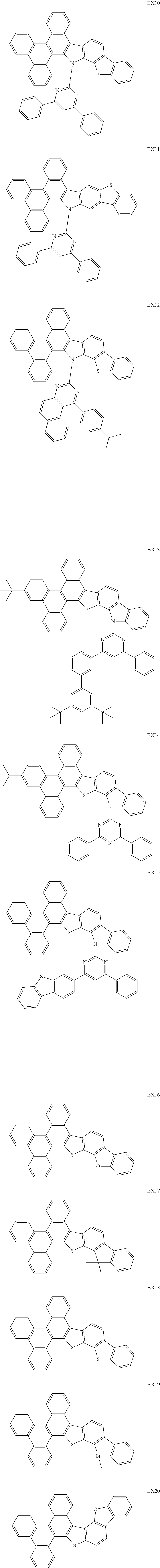

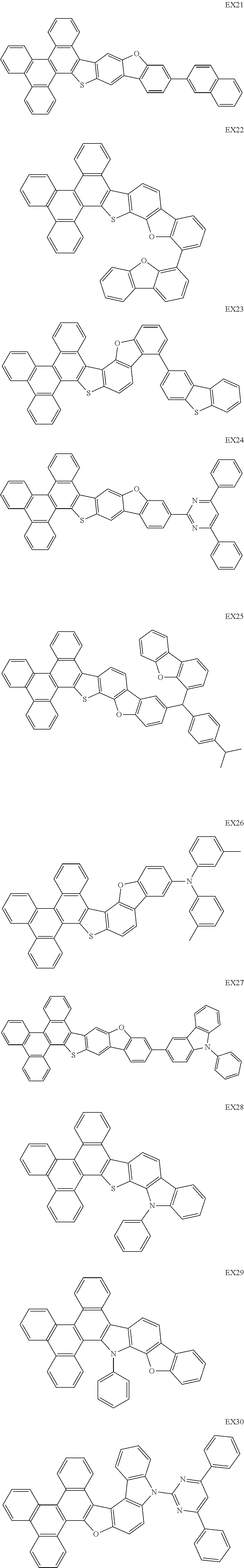

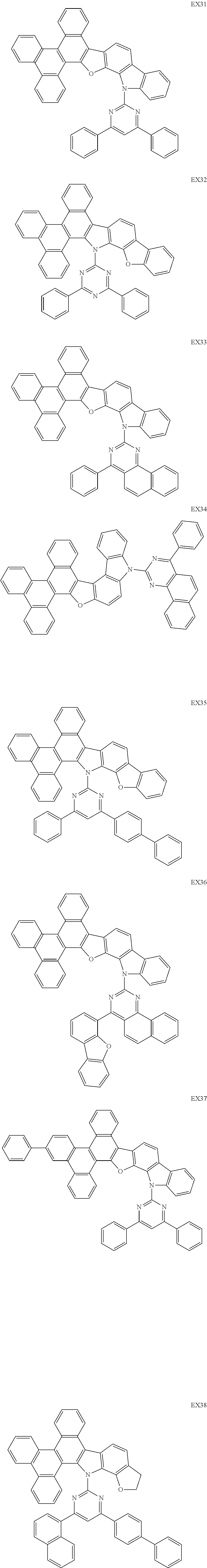

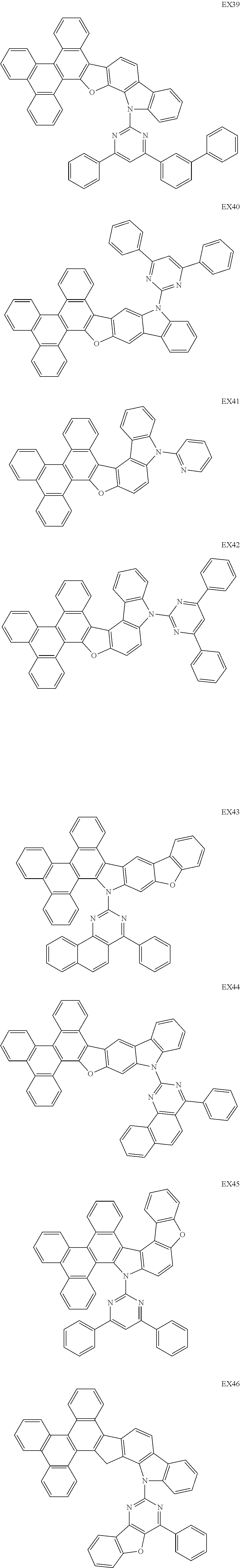

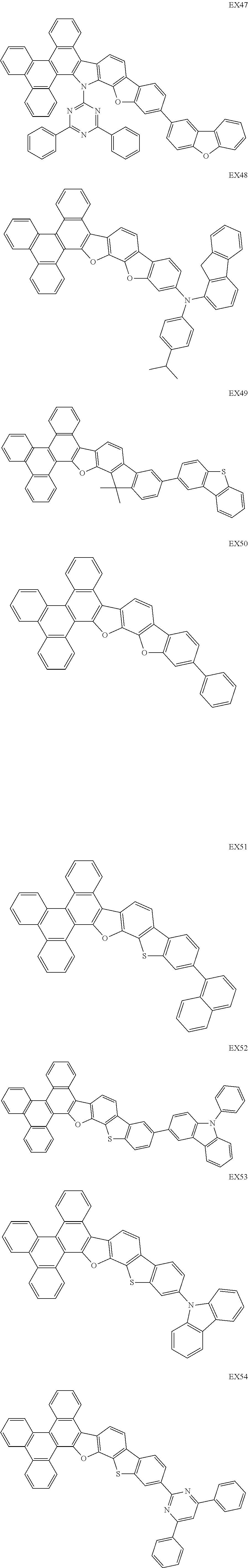

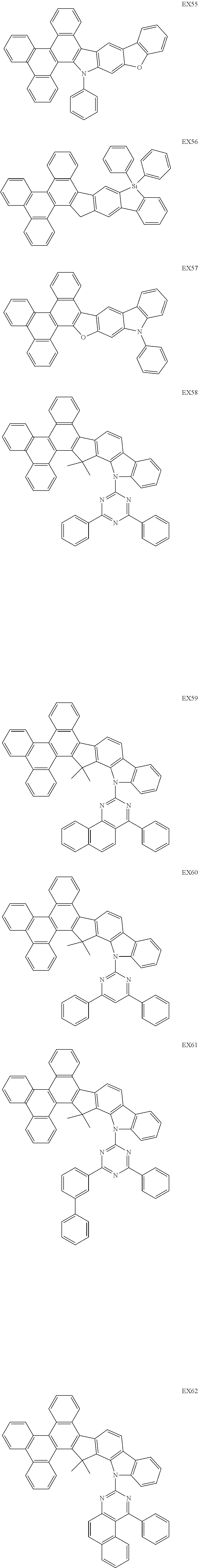

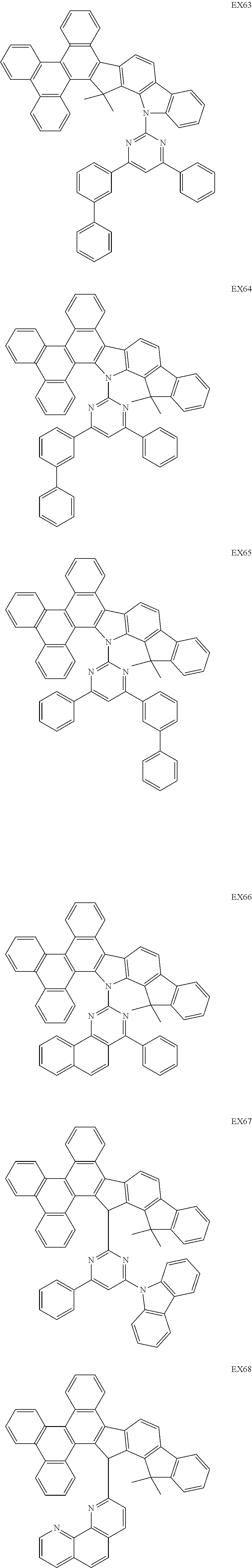

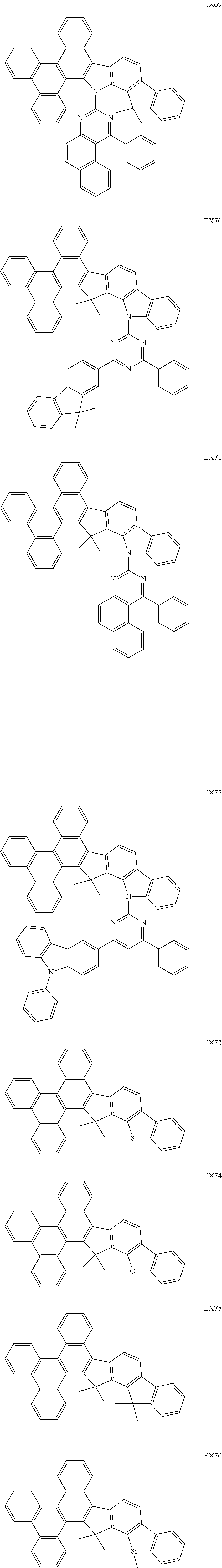

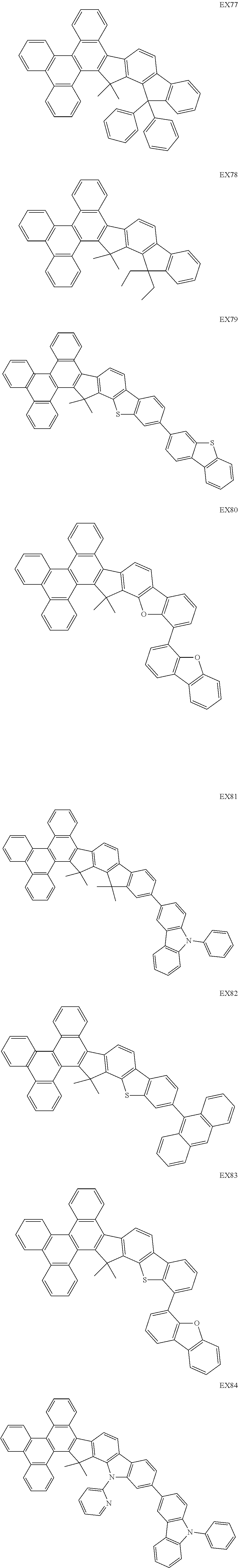

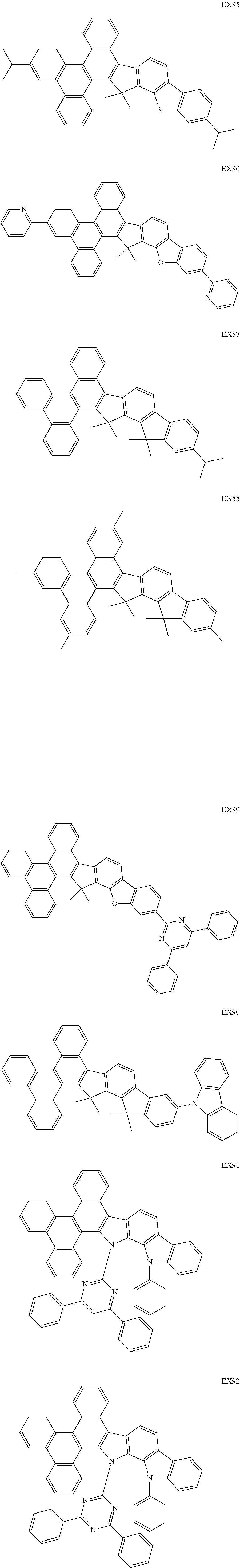

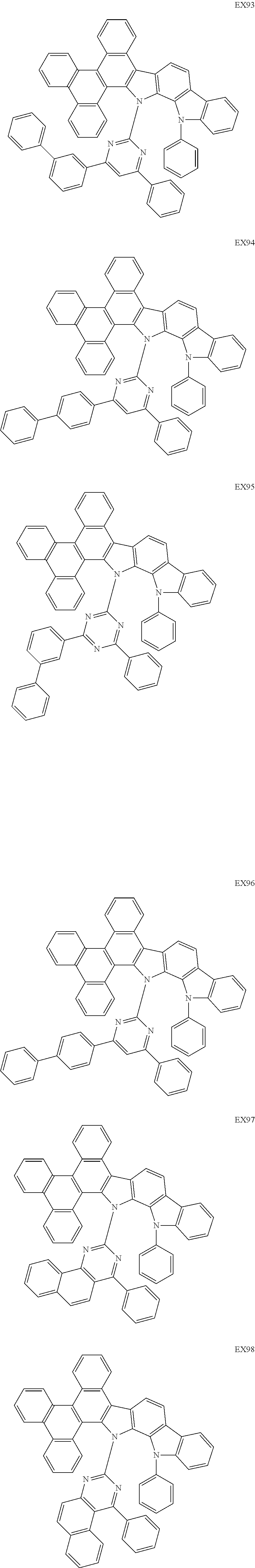

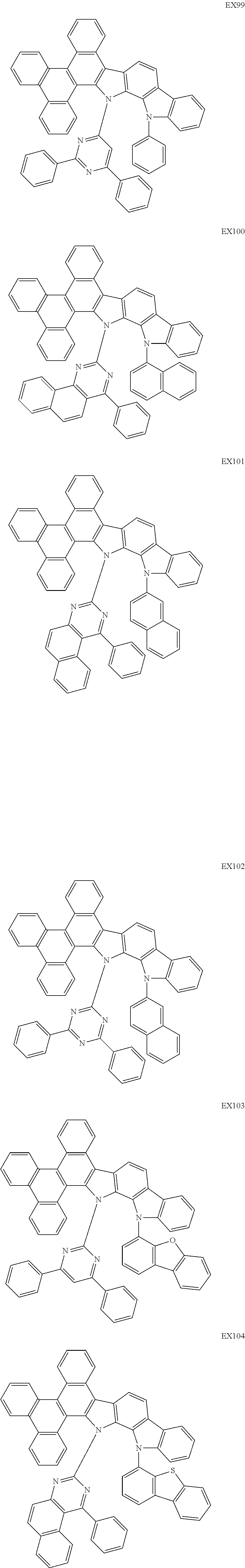

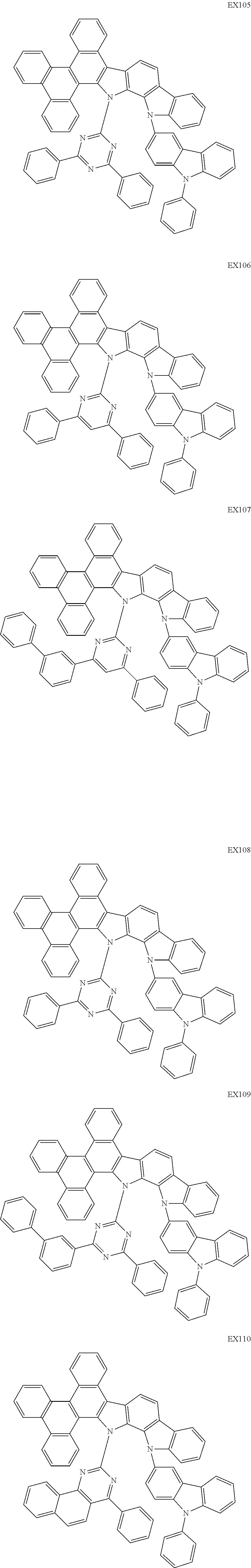

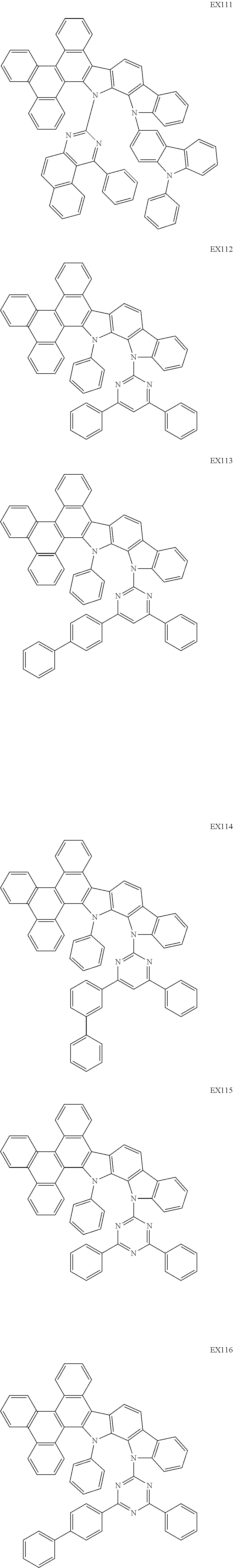

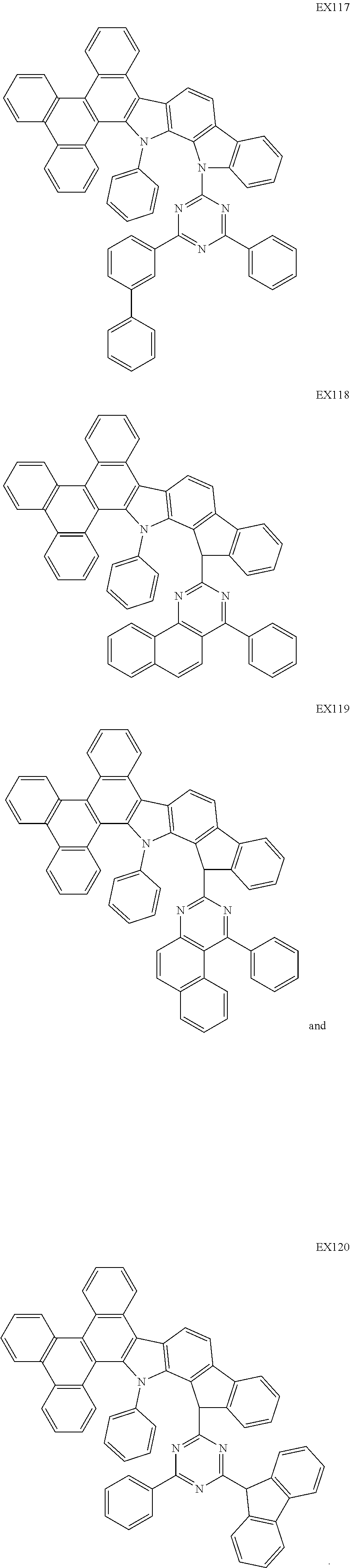 [SMILES: c1ccc(-c2cccc(-c3nc(-c4ccccc4)nc(-n4c5ccccc5c5ccc6c7c8ccccc8c8c9ccccc9c9ccccc9c8c7n(-c7ccccc7)c6c54)n3)c2)cc1 ; c1ccc(-c2nc(C3c4ccccc4-c4ccc5c6c7ccccc7c7c8ccccc8c8ccccc8c7c6n(-c6ccccc6)c5c43)nc3c2ccc2ccccc23)cc1 ; c1ccc(-c2nc(C3c4ccccc4-c4ccc5c6c7ccccc7c7c8ccccc8c8ccccc8c7c6n(-c6ccccc6)c5c43)nc3ccc4ccccc4c23)cc1 ; c1ccc(-c2nc(C3c4ccccc4-c4ccccc43)nc(C3c4ccccc4-c4ccc5c6c7ccccc7c7c8ccccc8c8ccccc8c7c6n(-c6ccccc6)c5c43)n2)cc1]

In some embodiments, the organic compound is represented by the following formula (I):

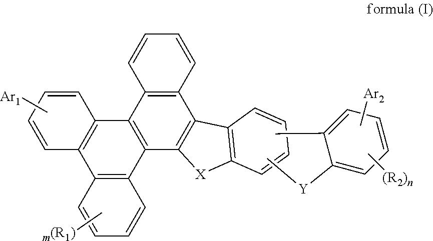

wherein X and Y are independently O, S, NR, C(CH₃)₂, C(CH₂CH₃)₂, Si(CH₃)₂ or Si(CH₂CH₃)₂;

wherein R is selected from the group consisting of aryl, aza-aryl, and combinations thereof;
wherein R is optionally substituted with phenyl, biphenyl, tricyclic aryl or tricyclic aza-aryl;
wherein Ar₁ and Ar₂ are independently absent or selected from the group consisting of methyl, isopropyl, isobutyl, phenyl, pyridinyl, and combinations thereof;
wherein m is an integer of 0, 1, 2, 3, 4, 5, 6, 7, 8, 9, 10 or 11; and
wherein n is an integer of 0, 1, 2 or 3.

In some embodiments, the organic compound is represented by one of the following formula (1) to formula (10):

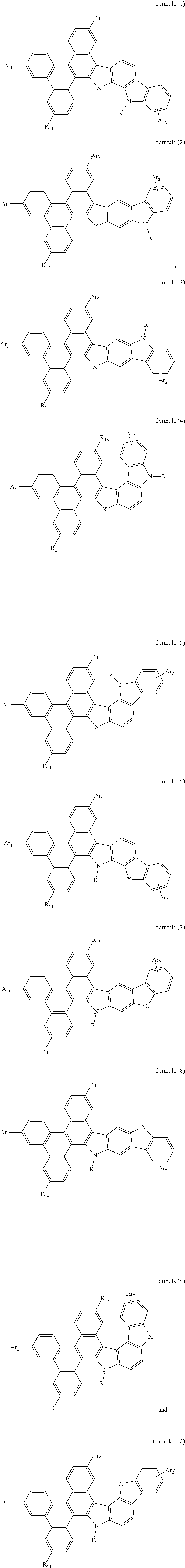

wherein Ar₁ and Ar₂ are independently absent or selected from the group consisting of methyl, isopropyl, isobutyl, phenyl, pyridinyl, and combinations thereof;
wherein X is O, S, C(CH₃)₂ or C(CH₂CH₃)₂;
wherein R is selected from the group consisting of aryl, aza-aryl, and combinations thereof;
wherein R is optionally substituted with phenyl, biphenyl, tricyclic aryl or tricyclic aza-aryl; and
wherein R₁₃ and R₁₄ are independently absent or methyl.

R may preferably be selected from the group consisting of monocyclic aryl, polycyclic aryl, monocyclic aza-aryl, polycyclic aza-aryl, and combinations thereof. More preferable, R is selected from the group consisting of monocyclic aryl, tricyclic aryl, monocyclic aza-aryl, tricyclic aza-aryl, and combinations thereof. The tricyclic aza-aryl may be selected from the group consisting of the following substituents:

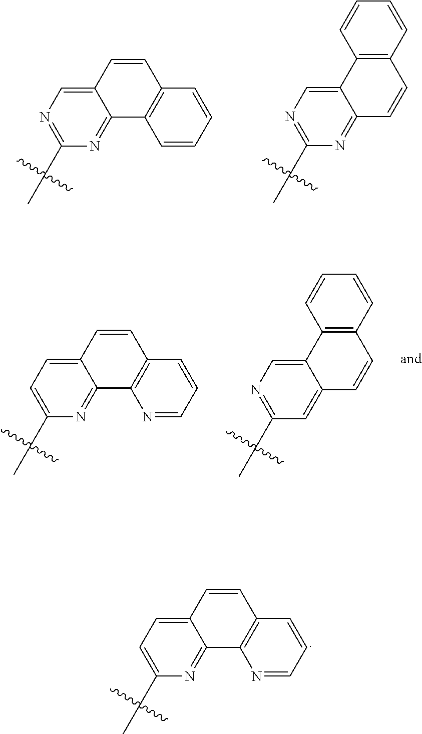 [SMILES: *c1ncc2ccc3ccccc3c2n1 ; *c1ncc2c(ccc3ccccc32)n1 ; *c1ccc2ccc3cccnc3c2n1 ; *c1cc2ccc3ccccc3c2cn1 ; *c1ccc2ccc3cccnc3c2n1]

The monocyclic aza-aryl may be selected from the group consisting of the following substituents:

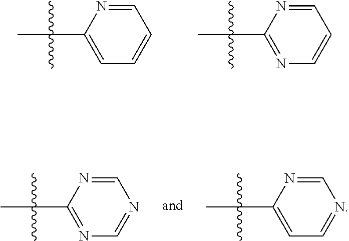 [SMILES: *c1ccccn1 ; *c1ncccn1 ; *c1ncncn1 ; *c1ccncn1]

Ar₂ may preferably be selected from the group consisting of the following substituents:

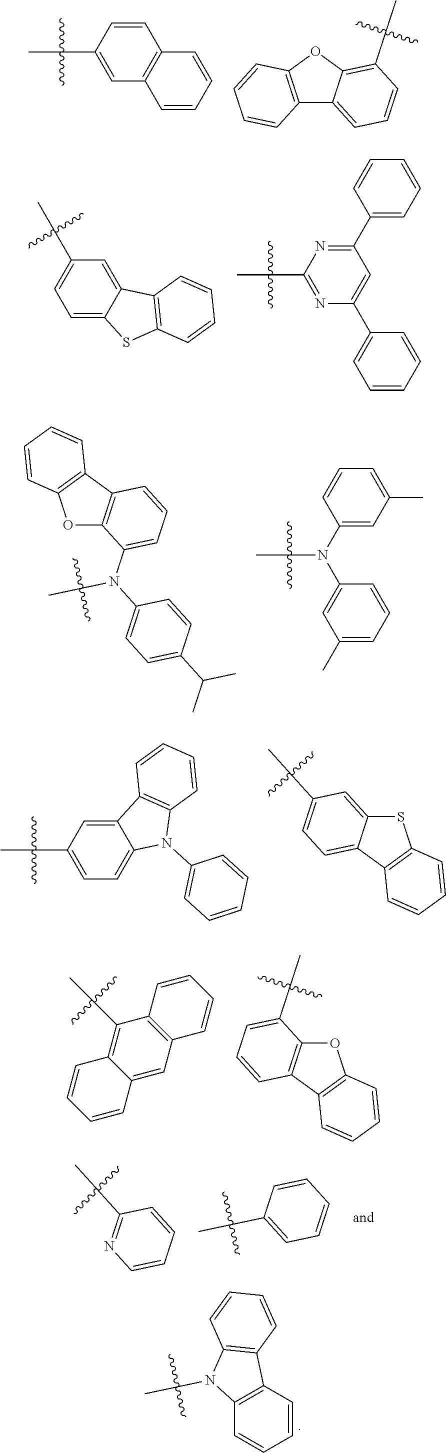

In another embodiment of the present invention, an organic electroluminescence device comprising an anode, a cathode and one or more organic layers formed between the anode and the cathode, wherein at least one of the organic layers comprises the organic compound of the present invention.

In some embodiments, the light emitting layer comprising the organic compound of the present invention is a host material. The host material may be a phosphorescent host material or a fluorescent host material.

In some embodiments, the organic thin film layer comprising the organic compound of the present invention is an electron transporting layer material.

In some embodiments, the organic thin film layer comprising the organic compound of the present invention is an hole blocking layer material.

In a further embodiment of the present invention, the organic electroluminescence device is a lighting panel. In other embodiment of the present invention, the organic electroluminescence device is a backlight panel.

The organic compound of the present invention, wherein one of the following is true:
X is N(Ar₃) if Y is S;
X is S if Y is N(Ar₄);
X is S if Y is C(R₇)(R₈);
X is S if Y is S;
X is S if Y is Si(R₉)(R₁₀);
X is S if Y is O;
X is O if Y is N(Ar₄);
X is N(Ar₃) if Y is O;
X is O if Y is O;
X is O if Y is C(R₇)(R₈);
X is O if Y is S;
X is C(R₃)(R₄) if Y is O;
X is O if Y is Si(R₉) (R₁₀);
X is C(R₃)(R₄) if Y is N(Ar₄);
X is N(Ar₃) if Y is C(R₇)(R₈);
X is C(R₃)(R₄) if Y is S;
X is C(R₃)(R₄) if Y is C(R₇)(R₈);
X is C(R₃)(R₄) if Y is Si(R₉)(R₁₀); and
X is N(Ar₃) if Y is N(Ar₄).

Detailed preparation of the organic compounds of the present invention will be clarified by exemplary embodiments below, but the present invention is not limited thereto. EXAMPLES 1 to 8 show the preparation of the organic compounds of the present invention, and EXAMPLES 9 to 10 show the fabrication and test reports of the organic EL devices.

### EXAMPLE 1

### Synthesis of Compound EX1

### Synthesis of Intermediate A1

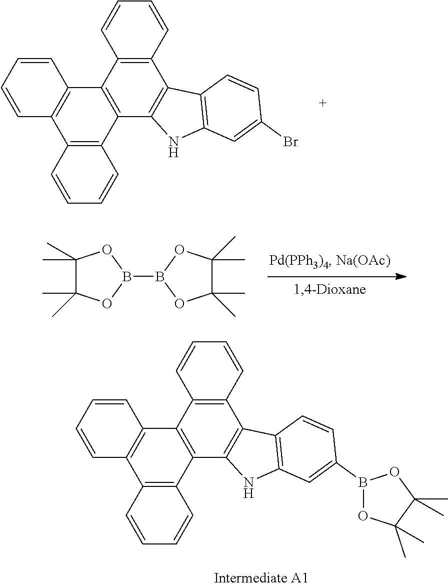

A mixture of 5 g (11.2 mmol) of 15-bromo-13H-benzo [c]phenanthro [9,10-a]carbazole (synthesis reference: U.S. Pat. No. 10,454,045), 3.1g (12.3 mmol) of 4,4,4′, 4′, 5,5,5′,5′,-octamethyl-2,2′-bi(1,3,2-dioxaborolane), 0.4g (0.34 mmol) of Pd(PPh₃)₄, 1.4 g (16.8 mmol) of sodium acetate, and 60 ml of 1,4-dioxane was degassed and placed under nitrogen, and then heated reflex for 6 hrs. After the reaction finished, the mixture was allowed to cool to room temperature. Subsequently, the organic layer was extracted with ethyl acetate and water, and then dried with anhydrous magnesium sulfate. After the solvent was removed, the residue was purified by column chromatography on silica to give Intermediate A1 (4.0 g, 72%).

### Synthesis of Intermediate A2

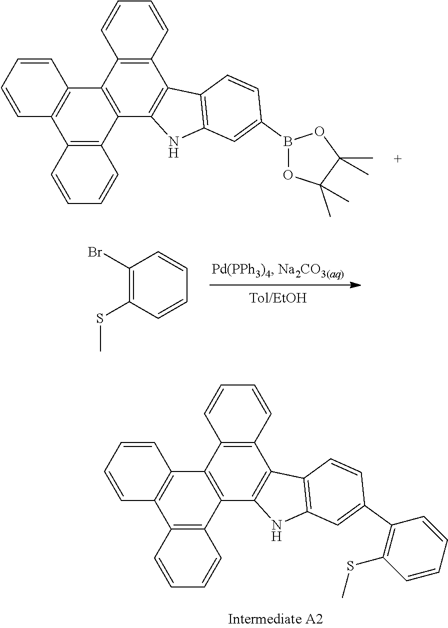

A mixture of 4.0 g (8.1 mmol) of Intermediate A1 1.6 g (8.1 mmol) of (2-bromophenyl)(methyl)sulfane, 0.23 g (0.2 mmol) of Pd(PPh₃)₄, 18 ml of 2M Na₂CO_{3(aq)}, 30 ml of EtOH, and 90 ml of toluene was degassed and placed under nitrogen, and then heated at 100° C. for 12hrs. After the reaction finished, the mixture was allowed to cool to room temperature. Subsequently, the reaction mixture was removed the solvent, then extracted with dichloromethane and water, and then dried with anhydrous MgSO₄. After the solvent was removed, the residue was purified by column chromatography on silica to give Intermediate A2 (3.0g, 76%).

### Synthesis of Intermediate A3

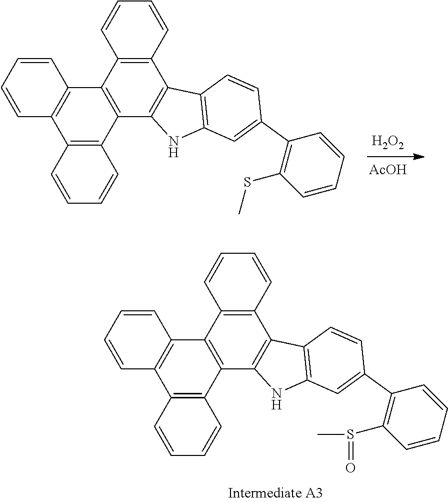

A mixture of 3.0g of Intermediate A2 (6.1 mmol), 30 ml of DCM and 60ml of Glacial acetic. To the mixture, 10ml of 35% H₂O₂ solution was added at 0° C. and the mixture was stirred for 18 h. The solution was extracted with Na₂SO₃ aqueous solution. The organic layer was dried with anhydrous magnesium sulfate and then the solvent was evaporated under reduced pressure, yielding Intermediate A3 (2.8 g, 95%).

### Synthesis of Intermediate A4

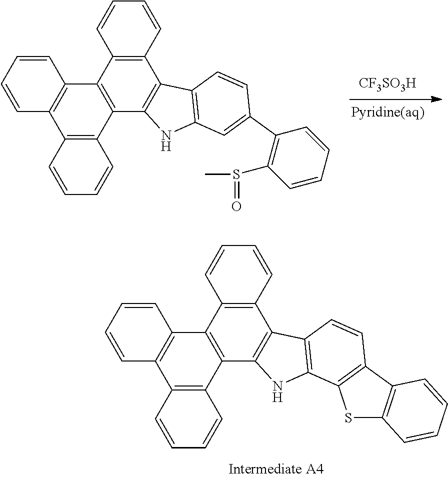

A mixture of 2.8g (5.8 mmol) of Intermediate A3, 26.1g (174 mol) of Trifluoromethanesulphonic acid was degassed and placed under nitrogen, and stir at room temperature for 48 h. After the reaction finished, 800ml of water/pyridine 5:1 was added and then heated under reflux for 20 min. After the reaction finished, the mixture was allowed to cool to room temperature. The solution was extracted with dichloromethane and water. The organic layer was dried with anhydrous magnesium sulfate and then the solvent was evaporated under reduced pressure. The pure product was purified by column chromatography on silica to give Intermediate A4 (0.4g, 15%).

### Synthesis of Compound EX1

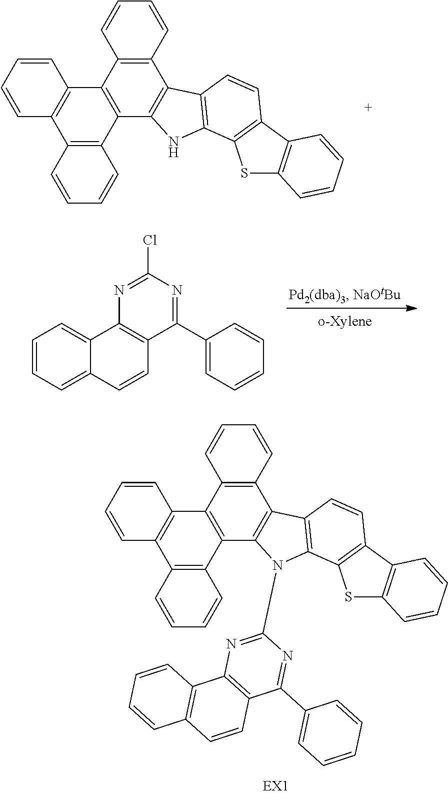

A mixture of 0.4g (0.87 mmole) of Intermediate A4, 0.28g (0.96 mmole) of 2-chloro-4-phenylbenzo[h]quinazoline, 0.08g (0.09 mmole) of Pd₂(dba)₃, 0.17g (1.74 mmole) of Sodium tert-butoxide, and 12 ml of o-Xylene was degassed and placed under nitrogen, and then heated at 150° C. for 16h. After the reaction finished, the mixture was allowed to cool to room temperature. Then 50 ml of MeOH was added while stirring and the precipitated product was filtered off with suction to obtain the product EX1 (0.35 g, 55%), which was recrystallized from EtOH. MS(m/z , FAB⁺): 727.9

### EXAMPLE 2 to 3

### Synthesis of Compound EX3 and EX10

### Synthesis of Compound EX3 and EX10 were prepared according to the synthesis method of Compound EX1.

| | | | Weight |
|---|---|---|---|
| Reactant structure | | Product structure | Yield |
| 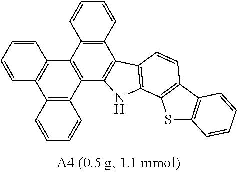 [SMILES: c1ccc2c(c1)sc1c2ccc2c1[nH]c1c2c2ccccc2c2c3ccccc3c3ccccc3c12] | 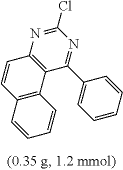 [SMILES: Clc1nc(-c2ccccc2)c2c(ccc3ccccc32)n1] | 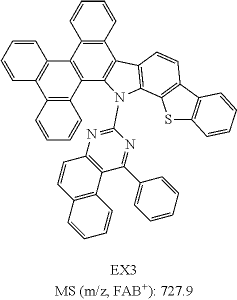 [SMILES: c1ccc(-c2nc(-n3c4c(ccc5c6ccccc6sc54)c4c5ccccc5c5c6ccccc6c6ccccc6c5c43)nc3ccc4ccccc4c23)cc1] | 0.4 g 52% |
| 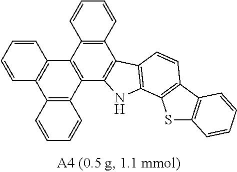 [SMILES: c1ccc2c(c1)sc1c2ccc2c1[nH]c1c2c2ccccc2c2c3ccccc3c3ccccc3c12] | 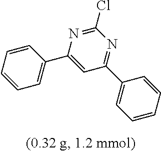 [SMILES: Clc1nc(-c2ccccc2)cc(-c2ccccc2)n1] | 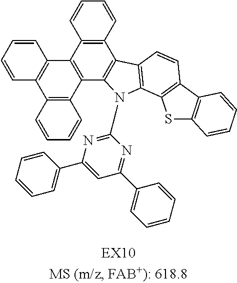 [SMILES: c1ccc(-c2cc(-c3ccccc3)nc(-n3c4c(ccc5c6ccccc6sc54)c4c5ccccc5c5c6ccccc6c6ccccc6c5c43)n2)cc1] | 0.4 g 62% |

### EXAMPLE 4

### Synthesis of Compound EX2

### Synthesis of Intermediate B1

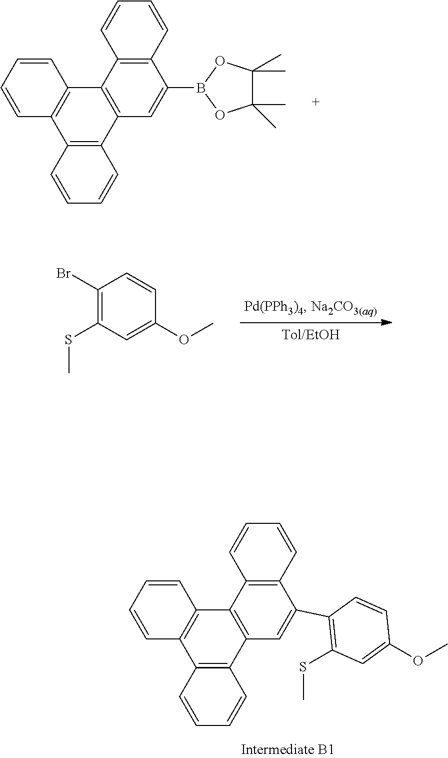 [SMILES: CC1(C)OB(c2cc3c4ccccc4c4ccccc4c3c3ccccc23)OC1(C)C ; COc1ccc(Br)c(SC)c1 ; COc1ccc(-c2cc3c4ccccc4c4ccccc4c3c3ccccc23)c(SC)c1]

A mixture of 5.0 g (12.4 mmol) of 2- (benzo [g] chrysen-10-yl) -4,4 ,5,5-tetramethyl-1,3,2-dioxaborolane (synthesis reference: U.S. Pat. No. 10,454,045), 3.2g (13.6 mmol) of (2-bromo-5-methoxyphenyl)(methyl)sulfane, 0.7g (0.62 mmol) of Pd(PPh₃)₄, 28ml of 2 M Na₂CO_{3(aq)}, 40 ml of EtOH, and 120 ml of toluene was degassed and placed under nitrogen, and then heated at 100° C. for 12hrs. After the reaction finished, the mixture was allowed to cool to room temperature. Subsequently, the reaction mixture was removed the solvent, then extracted with dichloromethane and water, and then dried with anhydrous MgSO₄. After the solvent was removed, the residue was purified by column chromatography on silica to give Intermediate B1 (2.8g, 52%).

### Synthesis of Intermediate B2

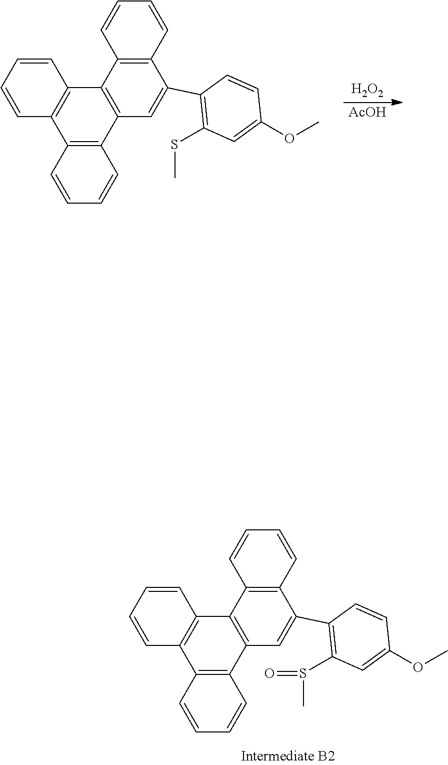

A mixture of 2.8g of Intermediate B1 (6.5 mmol), 30 ml of DCM and 60ml of Glacial acetic. To the mixture, 10ml of 35% H₂O₂ solution was added at 0° C. and the mixture was stirred for 18 h. The solution was extracted with Na₂SO₃ aqueous solution. The organic layer was dried with anhydrous magnesium sulfate and then the solvent was evaporated under reduced pressure, yielding Intermediate B2 (2.7g, 94%).

### Synthesis of Intermediate B3

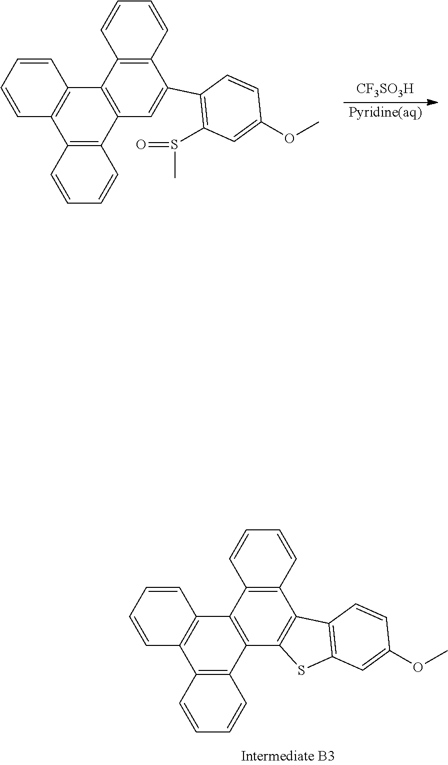 [SMILES: COc1ccc(-c2cc3c4ccccc4c4ccccc4c3c3ccccc23)c(S(C)=O)c1 ; "CF3SO3H" ; "Pyridine(aq)" ; COc1ccc2c(c1)sc1c2c2ccccc2c2c3ccccc3c3ccccc3c12]

A mixture of 2.7g (6.1 mmol) of Intermediate B2, 27.4 g (183 mol) of Trifluoromethanesulphonic acid was degassed and placed under nitrogen, and stir at room temperture for 48 h. After the reaction finished, 800 ml of water/pyridine 5:1 was added and then heated under reflux for 20 min. After the reaction finished, the mixture was allowed to cool to room temperature. The solution was extracted with dichloromethane and water. The organic layer was dried with anhydrous magnesium sulfate and then the solvent was evaporated under reduced pressure. The pure product was purified by column chromatography on silica to give Intermediate B3 (0.4 g, 16%).

### Synthesis of Intermediate B4

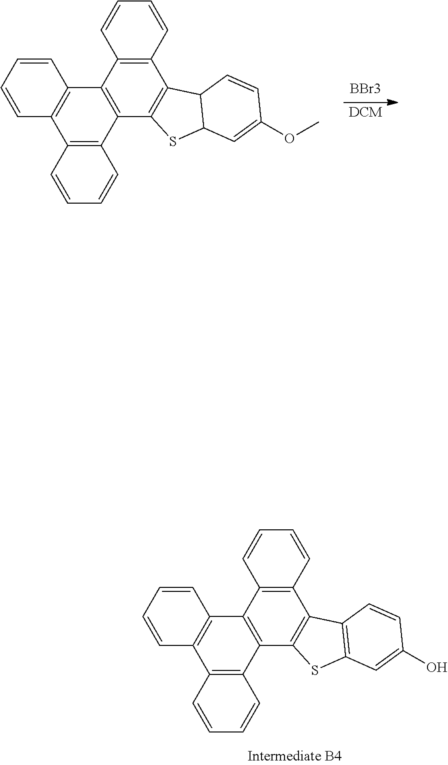

A mixture of 2 g (4.8 mmol) of Intermediate B3 and 60 ml of DCM were placed into the reactor under nitrogen. Boron tribromide (1 eq.) was added thereto and then stirred for 2 hrs until the reaction finished. The reaction mixture was extracted with dichloromethane and water, and then dried with anhydrous MgSO₄. The solvent was removed to give Intermediate B4 (1.7 g, 88%).

### Synthesis of Intermediate B5

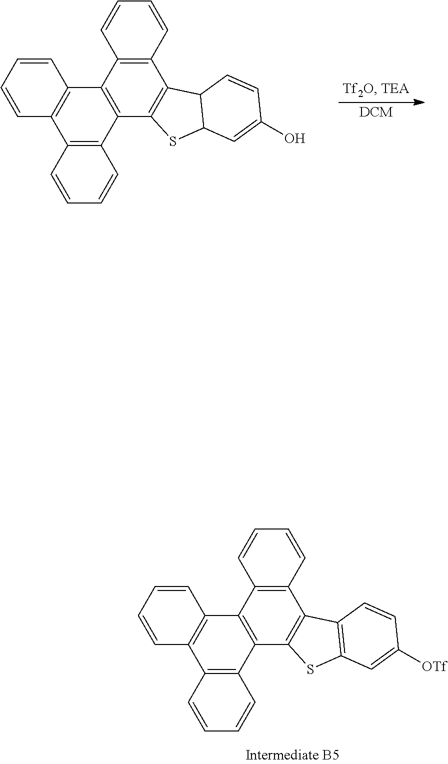

A mixture of 1.7 g (4.3 mmol) of Intermediate B4, 0.51 g (5.1 mmol) Triethylamine and 60 ml of DCM were placed into the reactor under nitrogen. Trifluoromethanesulfonic anhydride (1.1 equivalents) was slowly dropwised thereto at ice bath. and then stirred for overnight until the reaction finished. The reaction mixture was extracted with dichloromethane and water, and then dried with anhydrous MgSO₄. The solvent was removed to give Intermediate B5 (1.9 g, 86%).

### Synthesis of Intermediate B6

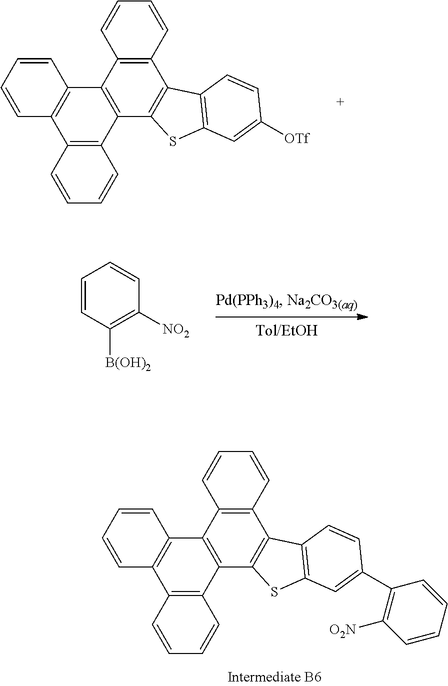 [SMILES: O=S(=O)(Oc1ccc2c(c1)sc1c2c2ccccc2c2c3ccccc3c3ccccc3c12)C(F)(F)F ; O=[N+]([O-])c1ccccc1B(O)O ; O=[N+]([O-])c1ccccc1-c1ccc2c(c1)sc1c2c2ccccc2c2c3ccccc3c3ccccc3c12]

A mixture of 1.9 g (3.7 mmol) of Intermediate B5, 0.67 g (4.1 mmol) of 2-nitrophenylboronic acid, 0.2 g (0.18 mmol) of Pd(PPh₃)₄, 11 ml of 2M Na₂CO_{3(aq)}, 20 ml of EtOH, and 60 ml of toluene was degassed and placed under nitrogen, and then heated at 100° C. for 12 hrs. After the reaction finished, the mixture was allowed to cool to room temperature. Subsequently, the reaction mixture was removed the solvent, then extracted with dichloromethane and water, and then dried with anhydrous MgSO₄. After the solvent was removed, the residue was purified by column chromatography on silica to give Intermediate B6 (1.2 g, 65%).

### Synthesis of Intermediate B7

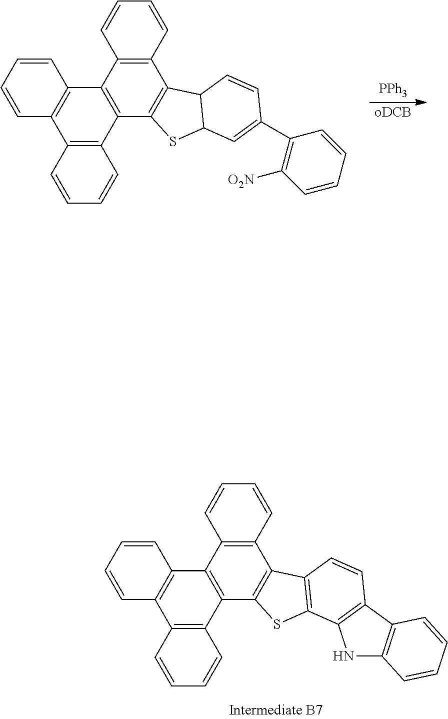 [SMILES: O=[N+]([O-])c1ccccc1C1=CC2Sc3c(c4ccccc4c4c5ccccc5c5ccccc5c34)C2C=C1 ; "oDCB" ; c1ccc2c(c1)[nH]c1c2ccc2c1sc1c2c2ccccc2c2c3ccccc3c3ccccc3c12 ; "PPh3"]

A mixture of 1.1 g (2.3 mmol) of Intermediate B6, 6.0 g (23 mmol) of Triphenylphosphine, and 40 ml of oDCB was placed under nitrogen gas, and then heated at 180° C. for 6 hrs. After the reaction finished, the mixture was allowed to cool to room temperature. The mixture was poured into water, and then filtered to give Intermediate B7 (0.8 g, 74%).

### Synthesis of Compound EX2

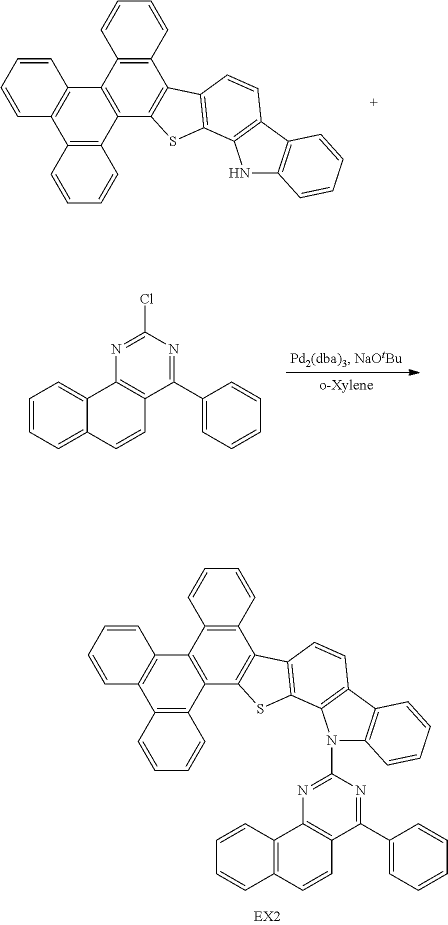 [SMILES: c1ccc2c(c1)[nH]c1c2ccc2c1sc1c2c2ccccc2c2c3ccccc3c3ccccc3c12 ; Clc1nc(-c2ccccc2)c2ccc3ccccc3c2n1 ; c1ccc(-c2nc(-n3c4ccccc4c4ccc5c(sc6c5c5ccccc5c5c7ccccc7c7ccccc7c65)c43)nc3c2ccc2ccccc23)cc1]

A mixture of 1 g (2.1 mmole) of Intermediate B7, 0.73 g (2.5 mmole) of 2-chloro-4-phenylbenzo[h]quinazoline, 0.1 g(0.1 mmole) of Pd₂(dba)₃, 0.3g(3.2 mmole) of Sodium tert-butoxide, and 30 ml of o-Xylene was degassed and placed under nitrogen, and then heated at 150° C. for 18 h. After the reaction finished, the mixture was allowed to cool to room temperature. Then 60 ml of MeOH was added while stirring and the precipitated product was filtered off with suction to obtain the product EX2 (1.1 g, 72%), which was recrystallized from EtOH. MS(m/z , FAB⁺): 727.9

### EXAMPLE 5 to 6

### Synthesis of Compound EX4 and EX5

### Synthesis of Compound EX4 and EX5 were prepared according to the synthesis method of Compound EX2.

| | | | Weight |
|---|---|---|---|
| Reactant structure | | Product structure | Yield |
| 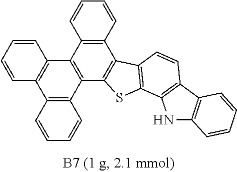 [SMILES: c1ccc2c(c1)[nH]c1c2ccc2c1sc1c2c2ccccc2c2c3ccccc3c3ccccc3c12] | 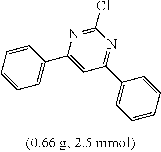 [SMILES: Clc1nc(-c2ccccc2)cc(-c2ccccc2)n1] | 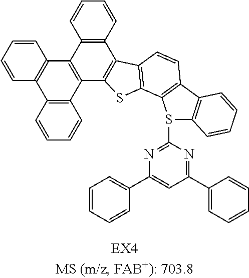 | 1.0 g 68% |
| 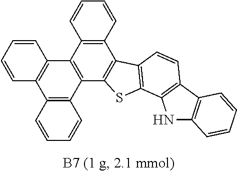 [SMILES: c1ccc2c(c1)[nH]c1c2ccc2c1sc1c2c2ccccc2c2c3ccccc3c3ccccc3c12] | 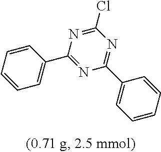 [SMILES: Clc1nc(-c2ccccc2)nc(-c2ccccc2)n1] | 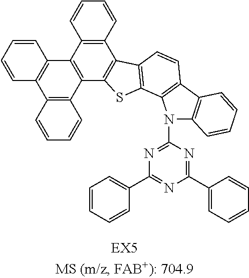 [SMILES: c1ccc(-c2nc(-c3ccccc3)nc(-n3c4ccccc4c4ccc5c(sc6c5c5ccccc5c5c7ccccc7c7ccccc7c65)c43)n2)cc1] | 0.93 g 63% |

### EXAMPLE 7

### Synthesis of Compound EX30

### Synthesis of Intermediate C1

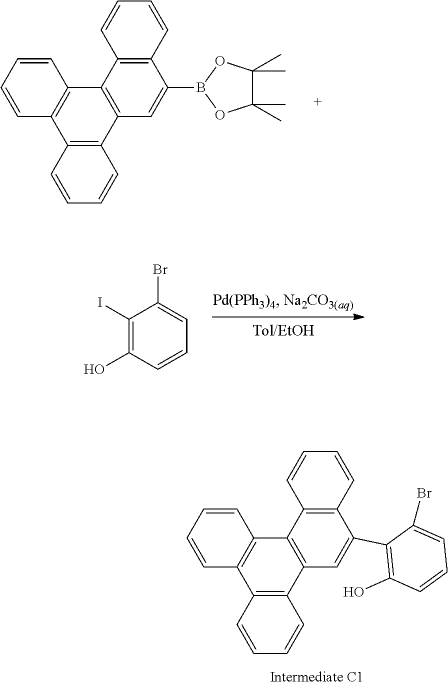 [SMILES: CC1(C)OB(c2cc3c4ccccc4c4ccccc4c3c3ccccc23)OC1(C)C ; Oc1cccc(Br)c1I ; Oc1cccc(Br)c1-c1cc2c3ccccc3c3ccccc3c2c2ccccc12]

A mixture of 5.0 g (12.4 mmol) of 2- (benzo [g] chrysen-10-yl) -4,4 ,5,5-tetramethyl-1,3,2-dioxaborolane (synthesis reference: U.S. Pat. No. 10,454,045), 4.1 g (13.6 mmol) of 3-bromo-2-iodophenol, 0.7 g (0.62 mmol) of Pd(PPh₃)₄, 28 ml of 2 M Na₂CO_{3(aq)}, 30 ml of EtOH, and 90 ml of toluene was degassed and placed under nitrogen, and then heated at 100° C. for 12hrs. After the reaction finished, the mixture was allowed to cool to room temperature. Subsequently, the reaction mixture was removed the solvent, then extracted with dichloromethane and water, and then dried with anhydrous MgSO₄. After the solvent was removed, the residue was purified by column chromatography on silica to give Intermediate C1 (3.8 g, 68%).

### Synthesis of Intermediate C2

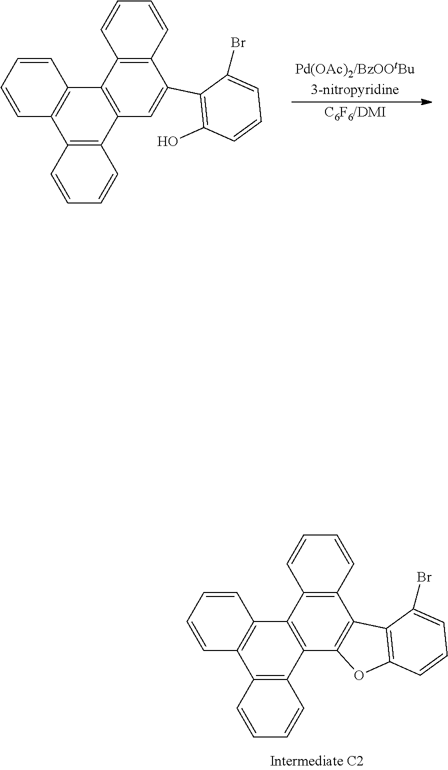 [SMILES: Oc1cccc(Br)c1-c1cc2c3ccccc3c3ccccc3c2c2ccccc12 ; "C6F6/DMI" ; "Pd(OAc)2/BzOOtBu3-nitropyridine" ; Brc1cccc2oc3c(c4ccccc4c4c5ccccc5c5ccccc5c34)c12]

A mixture of 3.8 g (8.4 mmol) of Intermediate C1, 0.19 g (0.84 mmol) of Pd(OAc)₂, 0.1g(0.84 mmol) of 3-nitropyridine, 3.3 g (16.8 mmol) of tert-butyl peroxybenzoate, 22 ml of C₆F₆, and 30 ml of DMI was degassed and placed under nitrogen, and then heated at 90° C. for 12hrs. Upon the completion of the reaction, the reaction product was extracted with CH₂Cl₂ and washed with water. After that, the extracted organic layer was dried with MgSO₄ and then concentrated. The concentrated resultant was separated by silica gel column chromatography to give Intermediate C2 (1.4g, 38%).

### Synthesis of Intermediate C3

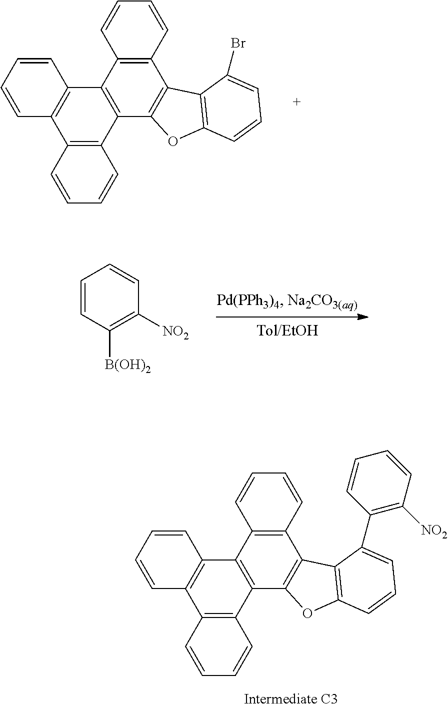 [SMILES: Brc1cccc2oc3c(c4ccccc4c4c5ccccc5c5ccccc5c34)c12 ; O=[N+]([O-])c1ccccc1B(O)O ; O=[N+]([O-])c1ccccc1-c1cccc2oc3c(c4ccccc4c4c5ccccc5c5ccccc5c34)c12]

A mixture of 1.4 g (3.1 mmol) of Intermediate C2, 0.62 g (3.7 mmol) of 2-nitrophenylboronic acid, 0.21 g (0.19 mmol) of Pd(PPh₃)₄, 9 ml of 2 M Na₂CO_{3 (aq)}, 20 ml of EtOH, and 60 ml of toluene was degassed and placed under nitrogen, and then heated at 100° C. for 12 hrs. After the reaction finished, the mixture was allowed to cool to room temperature. Subsequently, the reaction mixture was removed the solvent, then extracted with dichloromethane and water, and then dried with anhydrous MgSO₄. After the solvent was removed, the residue was purified by column chromatography on silica to give Intermediate C3 (1.1 g, 75%).

### Synthesis of Intermediate C4

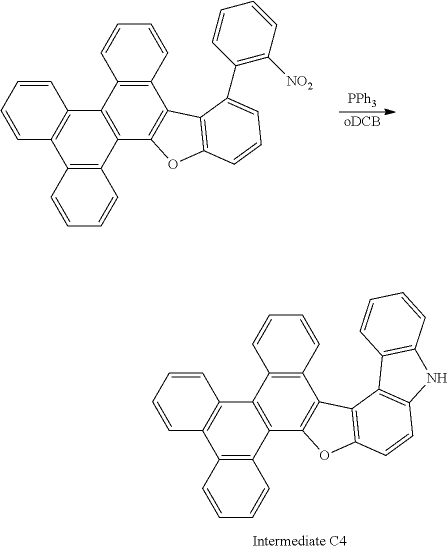 [SMILES: O=[N+]([O-])c1ccccc1-c1cccc2oc3c(c4ccccc4c4c5ccccc5c5ccccc5c34)c12 ; "oDCB" ; c1ccc2c(c1)[nH]c1ccc3oc4c5c6ccccc6c6ccccc6c5c5ccccc5c4c3c12 ; "PPh3"]

A mixture of 1.1 g (2.2 mmol) of Intermediate C3, 5.8 g (22 mmol) of Triphenylphosphine, and 35 ml of oDCB was placed under nitrogen gas, and then heated at 180° C. for 6 hrs. After the reaction finished, the mixture was allowed to cool to room temperature. The mixture was poured into water, and then filtered to give Intermediate C4 (0.8g, 78%).

### Synthesis of Compound EX30

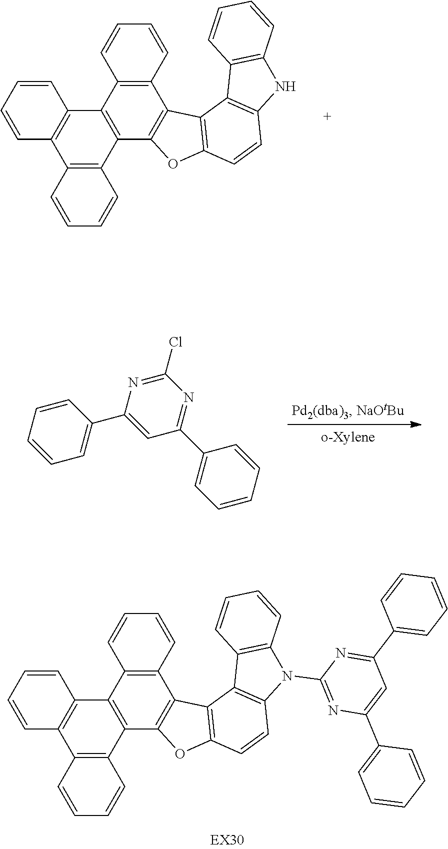 [SMILES: c1ccc2c(c1)[nH]c1ccc3oc4c5c6ccccc6c6ccccc6c5c5ccccc5c4c3c12 ; Clc1nc(-c2ccccc2)cc(-c2ccccc2)n1 ; c1ccc(-c2cc(-c3ccccc3)nc(-n3c4ccccc4c4c5c(ccc43)oc3c4c6ccccc6c6ccccc6c4c4ccccc4c35)n2)cc1]

A mixture of 0.8 g (1.7 mmole) of Intermediate C4, 0.54 g (2.0 mmole) of 2-chloro-4,6-diphenylpyrimidine, 0.08 g (0.08 mmole) of Pd₂(dba)₃, 0.25 g (2.6 mmole) of Sodium tert-butoxide, and 24 ml of o-Xylene was degassed and placed under nitrogen, and then heated at 150° C. for 18 h. After the reaction finished, the mixture was allowed to cool to room temperature. Then 60 ml of MeOH was added while stirring and the precipitated product was filtered off with suction to obtain the product EX30 (0.76 g, 65%), which was recrystallized from EtOH. MS(m/z, FAB⁺): 787.8

### EXAMPLE 8

### Synthesis of Compound EX34

### Synthesis of Compound EX34 were prepared according to the synthesis method of Compound EX30.

| | | | Weight |
|---|---|---|---|
| Reactant structure | | Product structure | Yield |
| 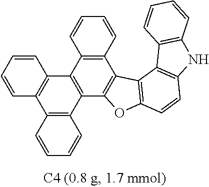 [SMILES: c1ccc2c(c1)[nH]c1ccc3oc4c5c6ccccc6c6ccccc6c5c5ccccc5c4c3c12] | 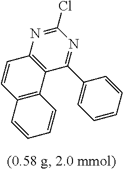 [SMILES: Clc1nc(-c2ccccc2)c2c(ccc3ccccc32)n1] | 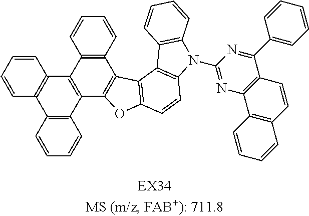 [SMILES: c1ccc(-c2nc(-n3c4ccccc4c4c5c(ccc43)oc3c4c6ccccc6c6ccccc6c4c4ccccc4c35)nc3c2ccc2ccccc23)cc1] | 0.85 g 70% |

### GENERAL METHOD OF PRODUCING ORGANIC EL DEVICE

ITO-coated glasses with 12 ohm/square in resistance and 120 nm in thickness are provided (hereinafter ITO substrate) and cleaned in a number of cleaning steps in an ultrasonic bath (e.g. detergent, deionized water). Before vapor deposition of the organic layers, cleaned ITO substrates are further treated by UV and ozone. All pre-treatment processes for ITO substrates are under clean room (class 100).

The organic layers are applied onto the ITO substrate in order by vapor deposition in a high-vacuum unit (10⁻⁷ Torr), such as: resistively heated quartz boats. The thickness of the respective layer and the vapor deposition rate (0.1˜0.3 nm/sec) are precisely monitored or set with the aid of a quartz-crystal monitor. It is also possible, as described above, for individual layers to consist of more than one compound, i.e. in general a host material doped with a dopant material. This is successfully achieved by co-vaporization from two or more sources, which means the organic compounds of the present invention are thermally stable.

Dipyrazino[2 ,3-f: 2,3-]quinoxaline-2,3,6 ,7,10,11-hexacarbonitrile (HAT-CN) is used to form the hole injection layer, and N,N-bis(naphthalene-1-yl)-N,N-bis(phenyl)-benzidine (NPB) is used to form the hole transporting layer of the organic EL device. 2,9-bis(naphthalene yl)-4,7-diphenyl-1,10-phenanthroline (NPhen) is used as the electron transporting material in organic EL device for its high thermal stability and long life-time than BPhen or BCP. For a phosphorescence emitting device, bis(2-methyl-8-quinolinolate)-4-(phenylphenolato)aluminium (BAlq) is used as the host material of emitting layer, and tris(1-phenylisoquinoline)-Iridium(III) (Ir(piq)₃) or tris(2-phenylquinoline)iridium(III) (Ir(2-phq)₃) is used as the dopant material. Compounds EX30, EX31, EX32, EX34, EX43, EX44, EX55, and EX57 are used as the electron transporting materials to compare with NPhen. Compounds EX1, EX2, EX3, EX4, EX5, EX6, and EX10 are used as the phosphorescent host materials to compare with BAlq. The chemical structures of conventional OLED materials and the exemplary organic compounds of the present invention for producing control and exemplary organic EL devices in this invention are shown as follows:

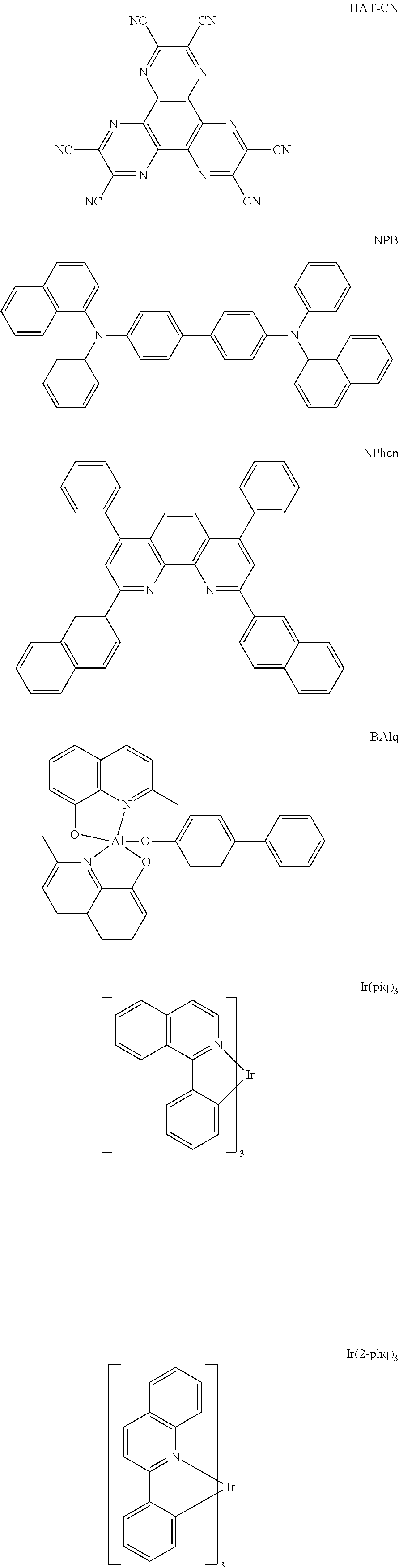

A typical organic EL device consists of low work function metals, such as Al, Mg, Ca, Li and K, as the cathode by thermal evaporation, and the low work function metals can help electrons injecting the electron transporting layer from cathode. In addition, for reducing the electron injection barrier and improving the organic EL device performance, a thin-film electron injecting layer is introduced between the cathode and the electron transporting layer. Conventional materials of electron injecting layer are metal halide or metal oxide with low work function, such as: LiF, MgO, or Li₂O.

On the other hand, after the organic EL device fabrication, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

### EXAMPLE 9

Using a procedure analogous to the above-mentioned general method, organic EL devices having the following device structure are produced (See the FIGURE): ITO/HAT-CN(20 nm)/NPB(50 nm)/Balq+10% Ir(2-phq)₃ (30 nm)/NPhen or EX30, EX31, EX32, EX34, EX43, EX44, EX55, or EX57 (30nm)/LiF(0.5 nm)/Al(160 nm). Each of the organic EL devices comprises a transparent electrode layer 6 (e.g., ITO), a hole injection layer 7, a hole transport layer 8, an emitting layer 9, an electron transport layer 10 and a metal electrode 11. The I-V-B and half-life time test reports of these yellow phosphorescence emitting organic EL devices are summarized in Table 1 below: the I-V-B is defined as at 1000 nits, and the half-life is defined as the time the initial luminance of 3000 cd/m² has dropped to half.

TABLE 1

| | Driving | Current | | |
|---|---|---|---|---|
| | Voltage | Efficiency | Device | Half-life |
| ETM | (V) | (cd/A) | color | (hrs) |
| NPhen | 6 | 14.32 | yellow | 475 |
| EX30 | 5.2 | 18.42 | yellow | 1088 |
| EX31 | 5.2 | 18.32 | yellow | 894 |
| EX32 | 5.4 | 17.78 | yellow | 734 |
| EX34 | 5.3 | 17.02 | yellow | 831 |
| EX43 | 5.4 | 17.12 | yellow | 805 |
| EX44 | 5.5 | 16.88 | yellow | 689 |
| EX55 | 5.6 | 17.44 | yellow | 764 |
| EX57 | 5.6 | 17.67 | yellow | 655 |

From the above test report summary of the organic EL devices, it is obvious that the organic compound of the present invention used as the electron transporting material exhibits better performance than the prior art material NPhen. In particular, the organic EL devices of the present invention employing the organic compound of the present invention as the electron transporting material to collocate with the host material Balq and the yellow dopant material Ir(2-phq)₃ have lower power consumption, higher current efficiency, and longer half-life time.

### EXAMPLE 10

Using a procedure analogous to the above-mentioned general method, organic EL devices emitting phosphorescence and having the following device structure were produced (See the FIGURE): ITO/HAT-CN(20 nm)/NPB(50 nm)/phosphorescent host (Balq or EX1, EX2, EX3, EX4, EX5, EX6, or EX10)+10% dopant (30 nm)/NPhen (30 nm)/LiF(0.5 nm)/A1(160 nm). Each of the organic EL devices comprises a transparent electrode layer 6 (e.g., ITO), a hole injection layer 7, a hole transport layer 8, an emitting layer 9, an electron transport layer 10 and a metal electrode 11. The I-V-B and half-life time test reports of these phosphorescence emitting organic EL devices are summarized in Table 2 below: the I-V-B is defined as at 6V, and the half-life time is defined as the time the initial luminance of 3000 cd/m² has dropped to half.

TABLE 2

| | Driving | Luminous | Current | | |
|---|---|---|---|---|---|
| Phosphorescent | Voltage | Efficiency | Efficiency | Device | Half-life |
| host + 10% dopant | (V) | (cd/m²) | (cd/A) | color | (hrs) |
| BAlq + Ir(piq)₃ | 6 | 673 | 8.16 | red | 454 |
| EX1 + Ir(piq)₃ | 6 | 883 | 10.62 | red | 832 |
| EX2 + Ir(piq)₃ | 6 | 863 | 10.32 | red | 808 |
| EX3 + Ir(piq)₃ | 6 | 815 | 9.88 | red | 659 |
| EX4 + Ir(piq)₃ | 6 | 835 | 10.03 | red | 689 |
| EX5 + Ir(piq)₃ | 6 | 831 | 10.01 | red | 629 |
| EX6 + Ir(piq)₃ | 6 | 844 | 9.98 | red | 713 |
| EX10 + Ir(piq)₃ | 6 | 871 | 10.42 | red | 753 |
| BAlq + Ir(2-phq)₃ | 6 | 1190 | 14.32 | yellow | 475 |
| EX1 + Ir(2-phq)₃ | 6 | 1838 | 22.13 | yellow | 868 |
| EX2 + Ir(2-phq)₃ | 6 | 1805 | 21.77 | yellow | 854 |
| EX3 + Ir(2-phq)₃ | 6 | 1625 | 19.58 | yellow | 843 |
| EX4 + Ir(2-phq)₃ | 6 | 1822 | 21.95 | yellow | 798 |
| EX5 + Ir(2-phq)₃ | 6 | 1801 | 21.63 | yellow | 835 |
| EX6 + Ir(2-phq)₃ | 6 | 1702 | 20.55 | yellow | 675 |
| EX10 + Ir(2-phq)₃ | 6 | 1812 | 21.69 | yellow | 762 |

From the above test report summary of the organic EL devices, it is obvious that the organic compound of the present invention used as the phosphorescent host material exhibits better performance than the prior art material BAlq. In particular, the organic EL devices of the present invention employing the organic compound of the present invention as the phosphorescent host material to collocate with the dopant material Ir(piq)₃ or Ir(2-phq)₃ have higher luminance and current efficiency and longer half-life time under the same voltage.

Accordingly, each of the organic EL devices, may also a red, amber or yellow electroluminescence device, may serve as a tail light or a turn light of a car having longer half-life, higher current efficiency, higher luminous efficiency and/or lower driving voltage.

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

What is claimed is:

1. An organic compound represented by the following formula (I): 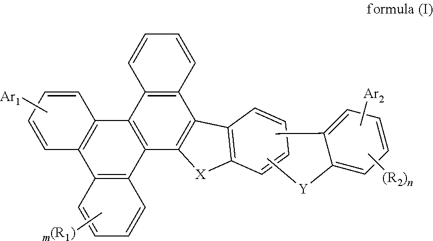
wherein m is an integer of 0 to 11;
wherein n is an integer of 0 to 3;
wherein X is O, S, C(R₃)(R₄), N(Ar₃), or Si(R₅)(R₆);
wherein Y is O, S, C(R₇)(R₈), N(Ar₄), or Si(R₉)(R₁₀);
wherein Ar₁ and Ar₂ are each independently a hydrogen atom, a halide, a substituted or unsubstituted arylamine group having 5 to 50 ring atoms, a substituted or unsubstituted heteroarylamine group having 5 to 50 ring atoms, a substituted or unsubstituted aryl group having 5 to 50 ring atoms, or a substituted or unsubstituted heteroaryl group having 5 to 50 ring atoms;
wherein Ar₃ and Ar₄ are each independently a hydrogen atom, a substituted or unsubstituted aryl group having 5 to 50 ring atoms, or a substituted or unsubstituted heteroaryl group having 5 to 50 ring atoms; and
wherein R₁ to R₁₀ are independently selected from the group consisting of a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, and a substituted or unsubstituted heteroaryl group having 3 to 30 carbon atoms.

2. The organic compound of claim 1, wherein Ar₁ and Ar₂ are independently selected from the group consisting of the following substituents: 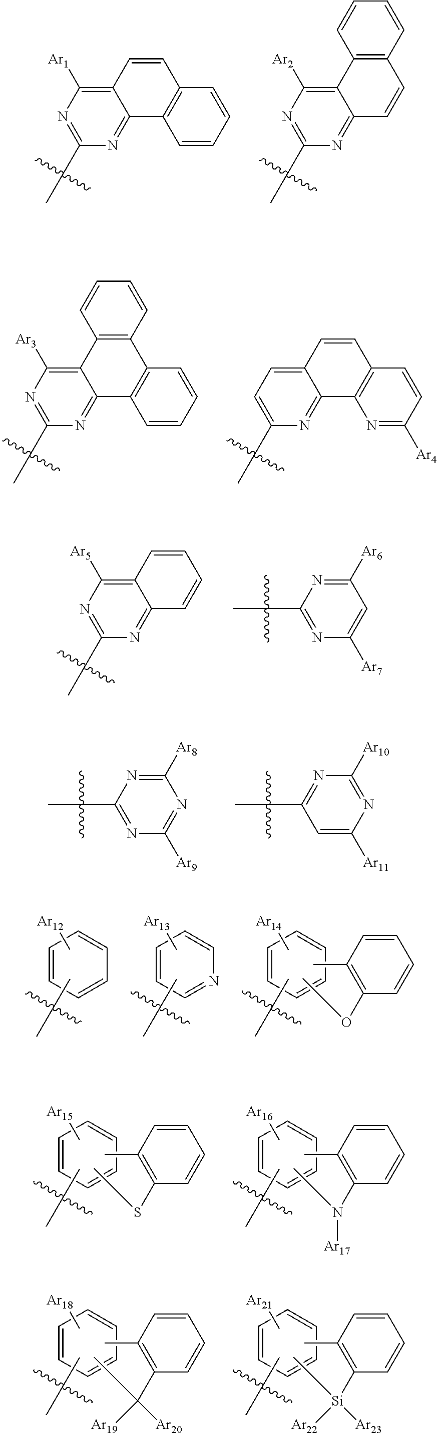 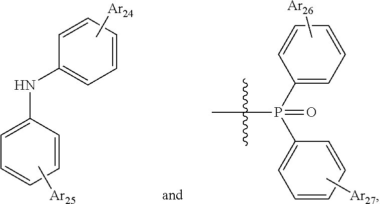
and wherein Ar₅˜Ar₂₇ are each independently a hydrogen atom, a substituted or unsubstituted aryl group having 5 to 50 ring atoms, or a substituted or unsubstituted heteroaryl group having 5 to 50 ring atoms.

3. The organic compound of claim 2, wherein Ar₁ and Ar₂ are independently selected from the group consisting of the following substituents:

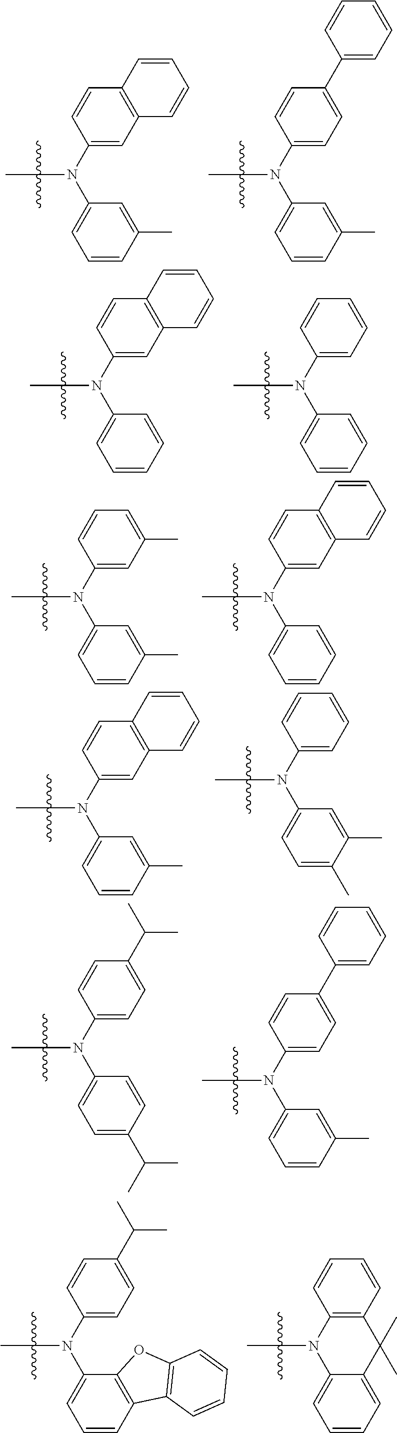

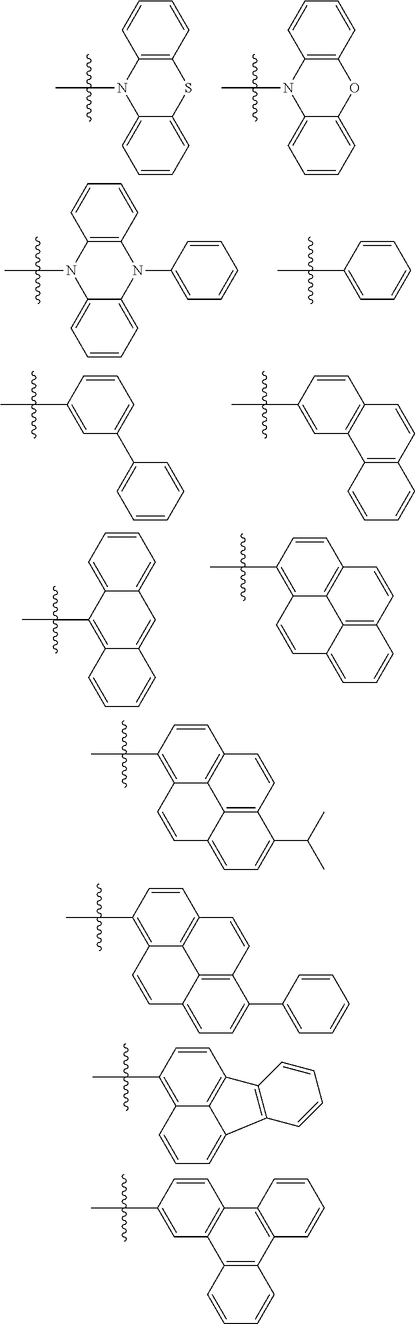

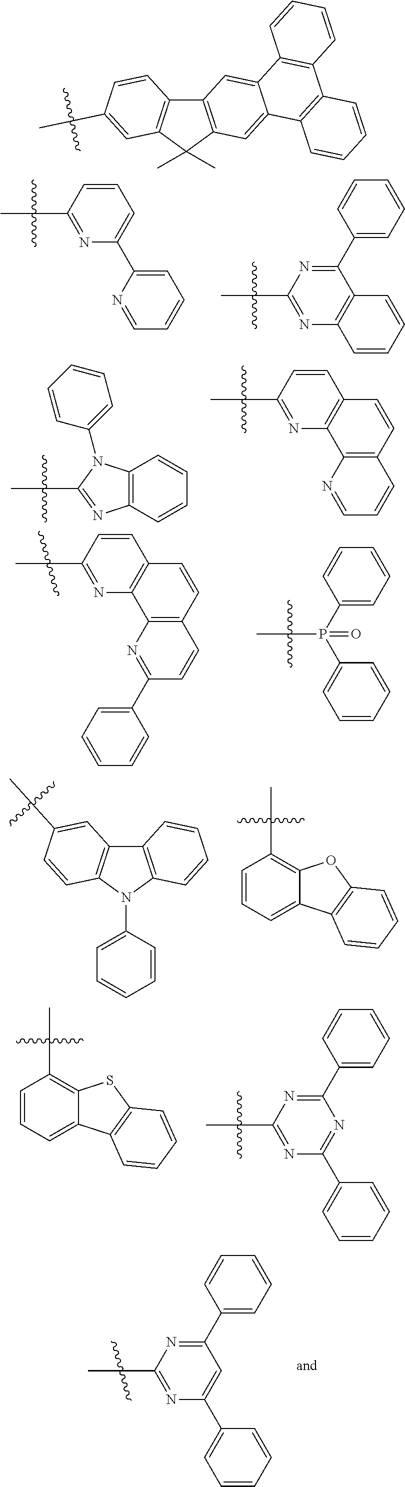

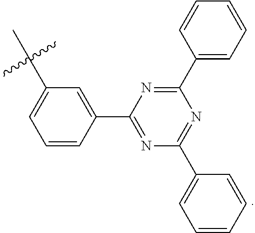 [SMILES: *c1cccc(-c2nc(-c3ccccc3)nc(-c3ccccc3)n2)c1]

4. The organic compound of claim 1, wherein Ar₃ and Ar₄ are independently selected from the group consisting of the following substituents: 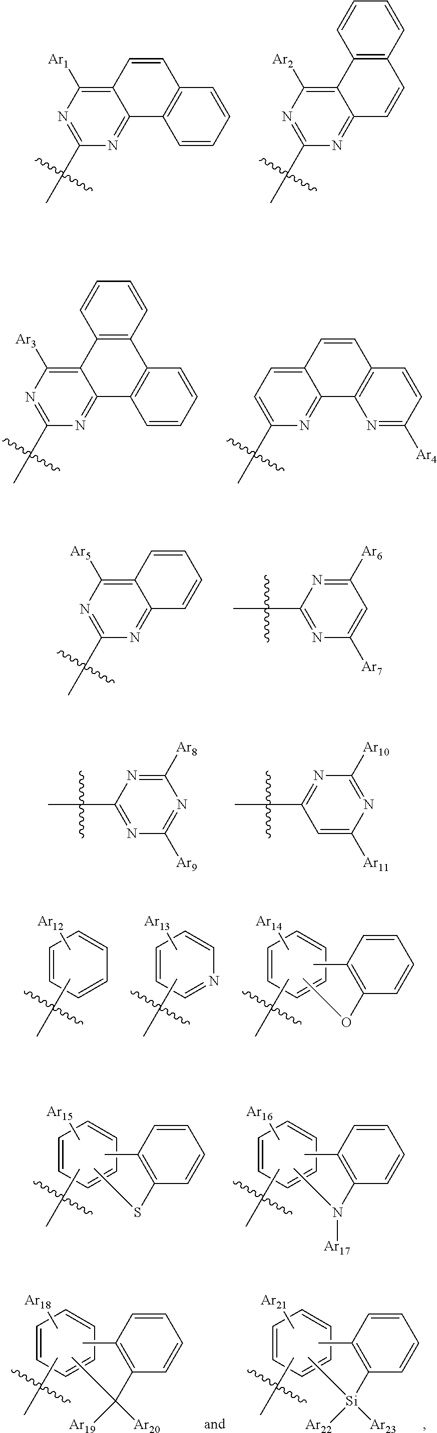
and wherein Ar₅˜Ar₂₃ are each independently a hydrogen atom, a substituted or unsubstituted aryl group having 5 to 50 ring atoms, or a substituted or unsubstituted heteroaryl group having 5 to 50 ring atoms.

5. According to claim 3, wherein Ar₃ and Ar₄ are independently selected from the group consisting of the following substituents:

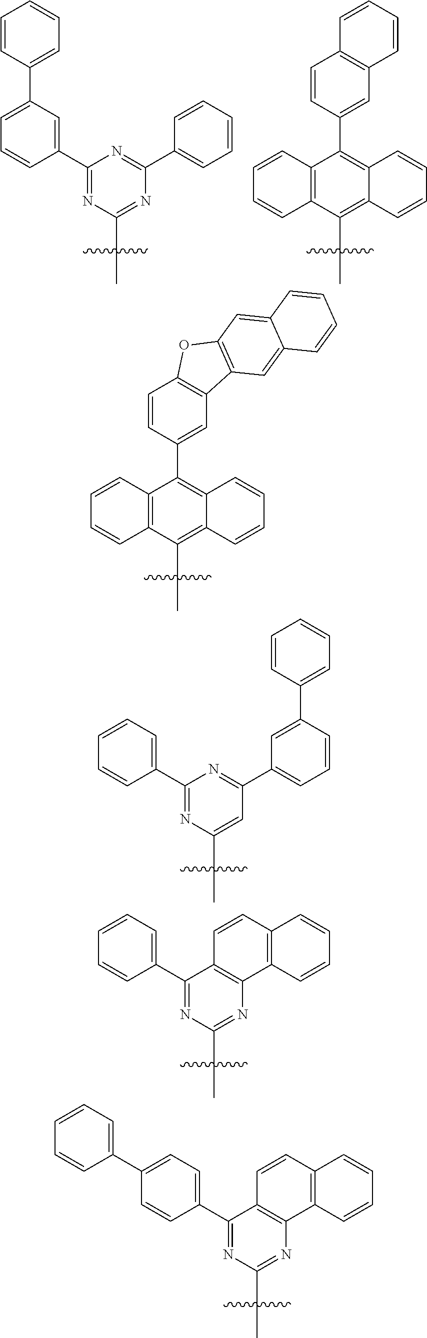

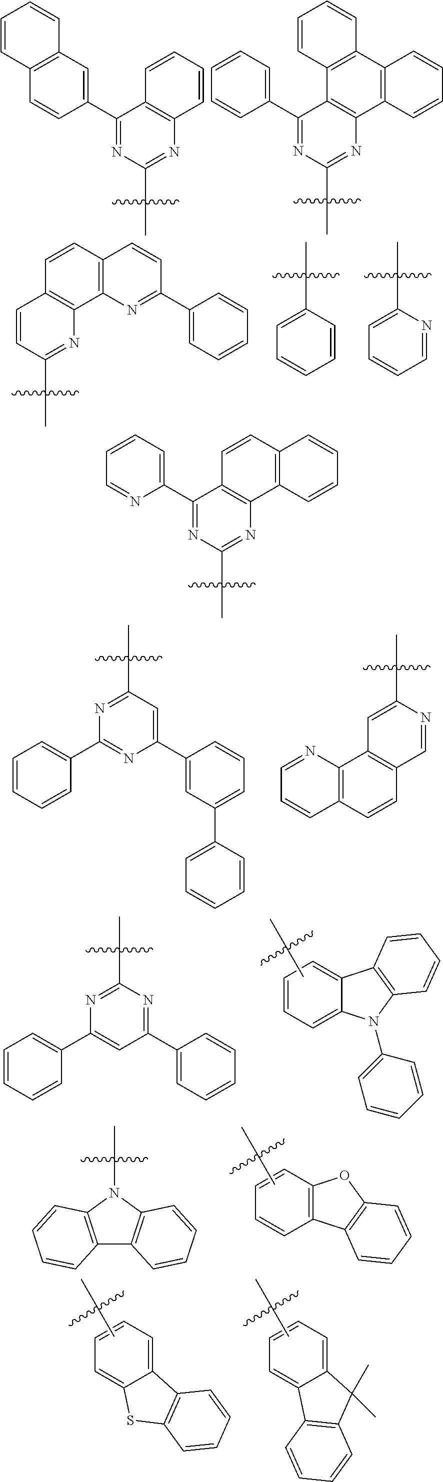

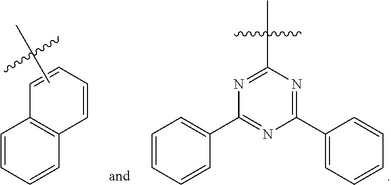

6. According to claim 1, wherein the organic compound is selected from the group consisting of the following compounds:

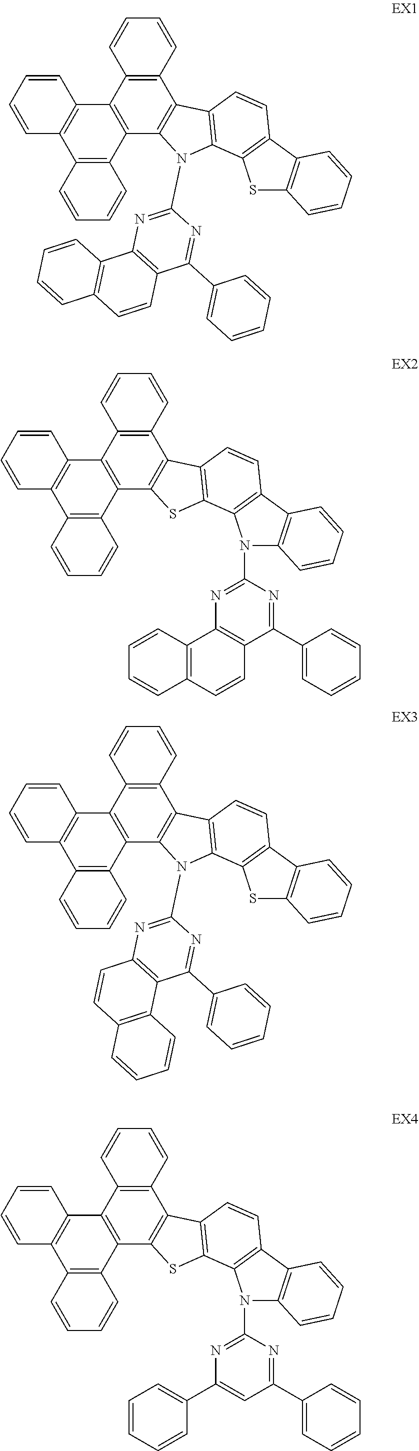

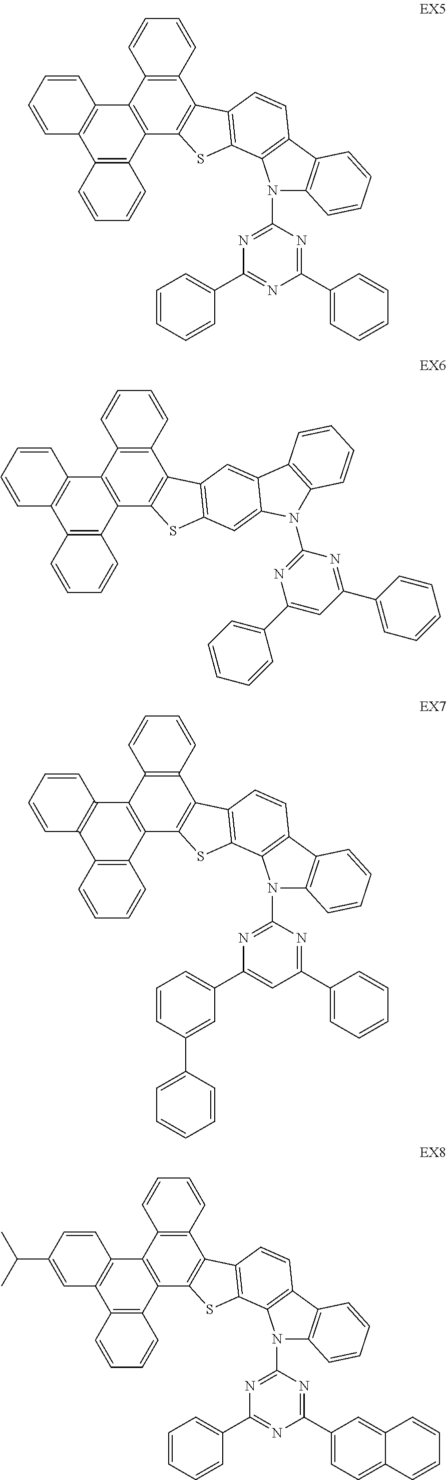

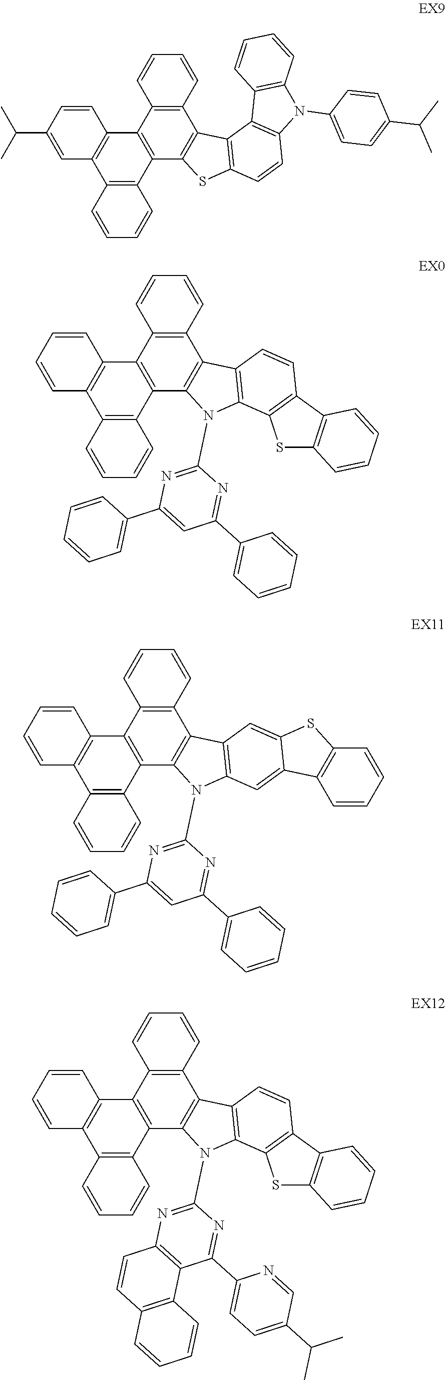

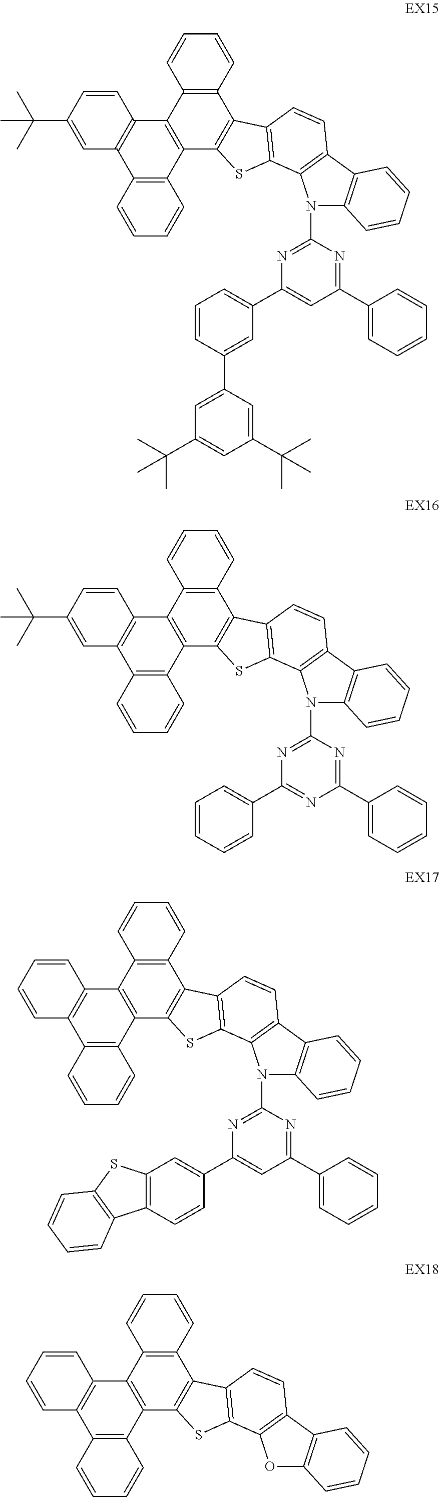

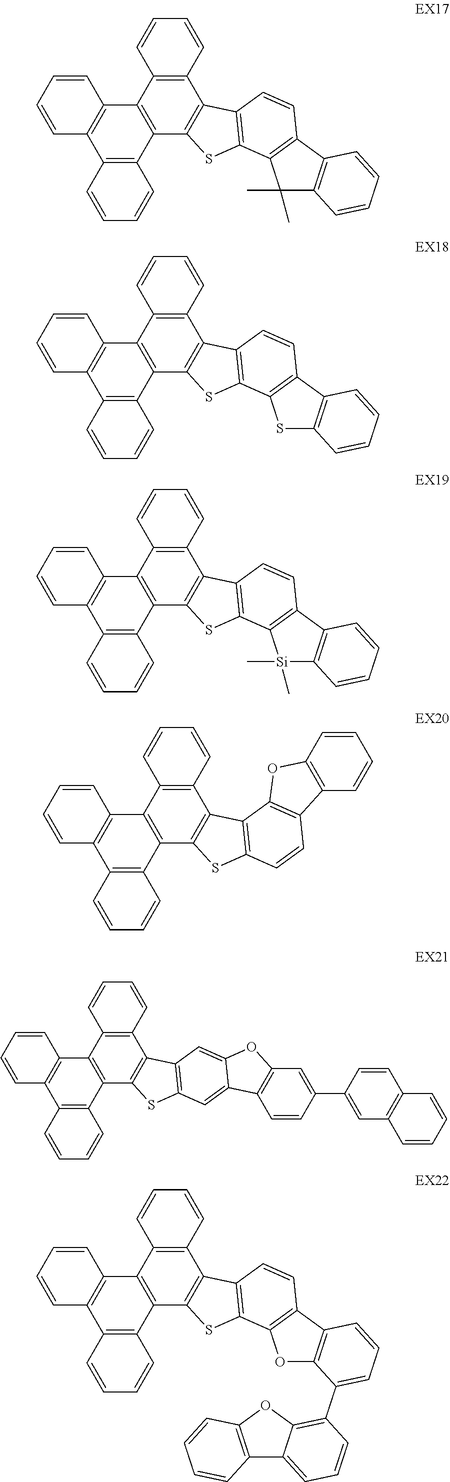

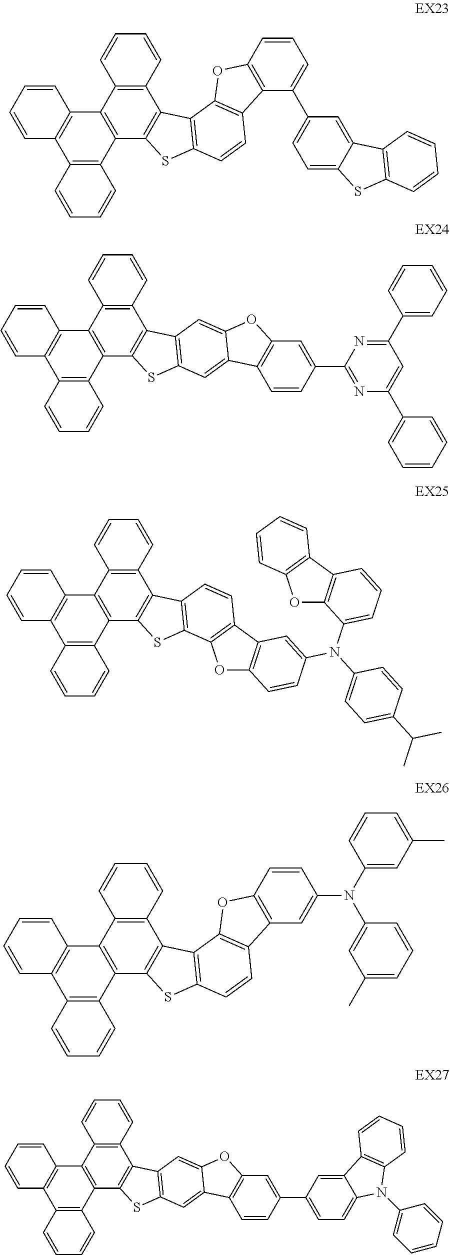

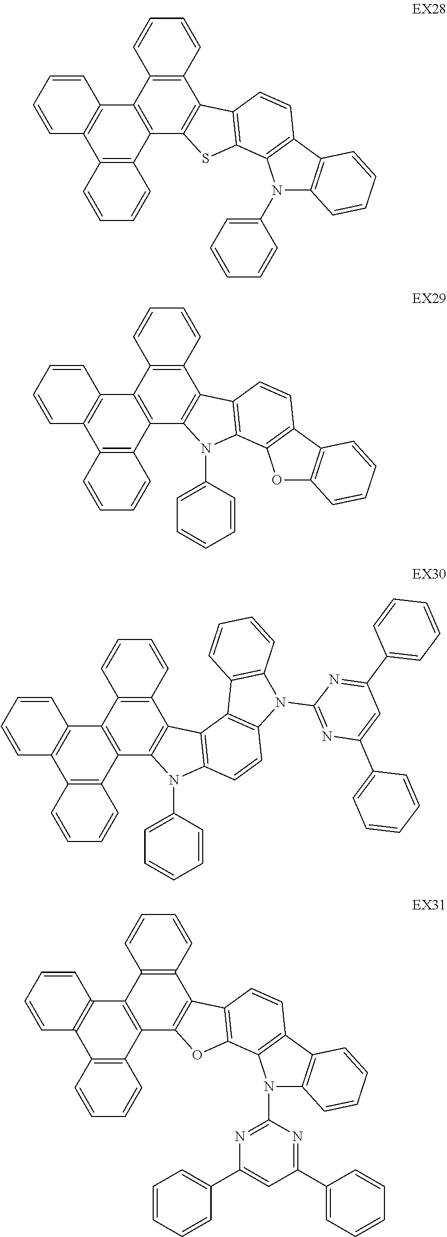

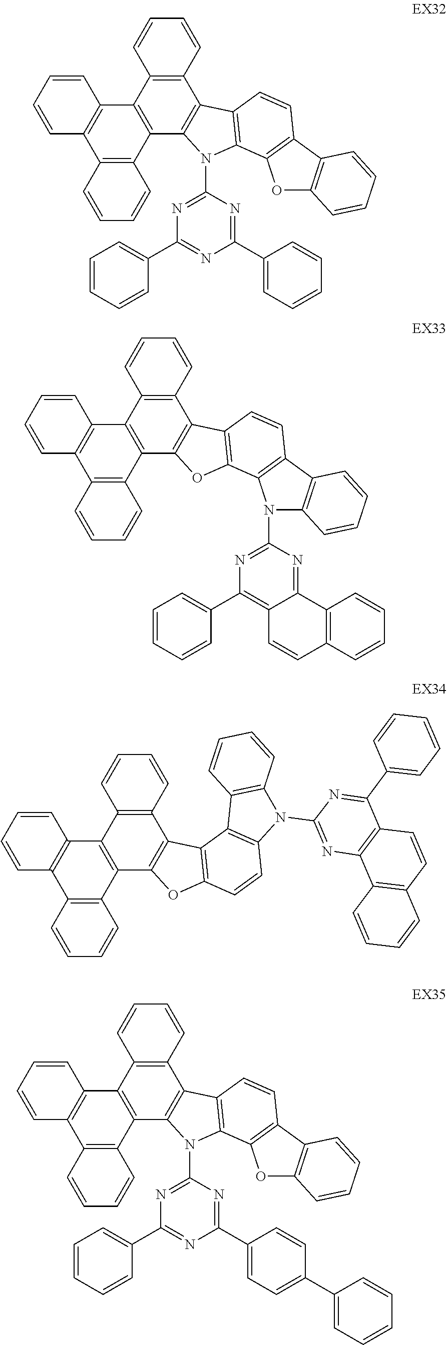

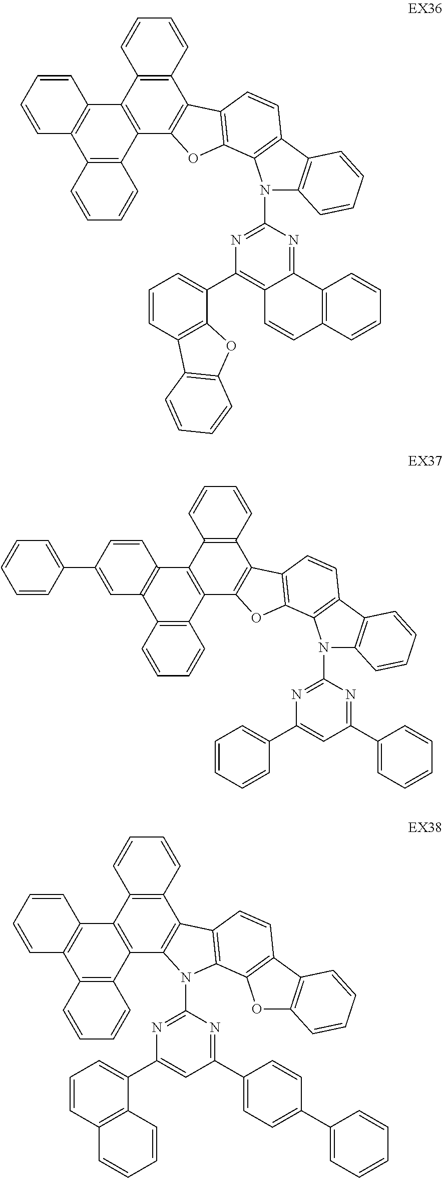 [SMILES: c1ccc2c(c1)ccc1c(-c3cccc4c3oc3ccccc34)nc(-n3c4ccccc4c4ccc5c(oc6c5c5ccccc5c5c7ccccc7c7ccccc7c65)c43)nc12 ; c1ccc(-c2ccc3c(c2)c2ccccc2c2c4oc5c(ccc6c7ccccc7n(-c7nc(-c8ccccc8)cc(-c8ccccc8)n7)c65)c4c4ccccc4c32)cc1 ; c1ccc(-c2ccc(-c3cc(-c4cccc5ccccc45)nc(-n4c5c(ccc6c7ccccc7oc65)c5c6ccccc6c6c7ccccc7c7ccccc7c6c54)n3)cc2)cc1]

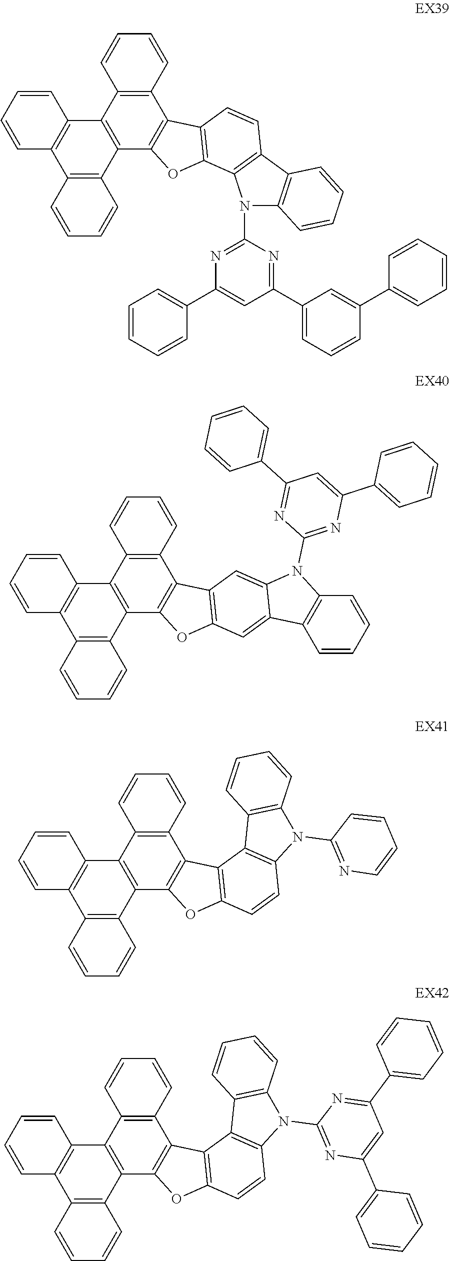 [SMILES: c1ccc(-c2cccc(-c3cc(-c4ccccc4)nc(-n4c5ccccc5c5ccc6c(oc7c6c6ccccc6c6c8ccccc8c8ccccc8c76)c54)n3)c2)cc1 ; c1ccc(-c2cc(-c3ccccc3)nc(-n3c4ccccc4c4cc5oc6c(c5cc43)c3ccccc3c3c4ccccc4c4ccccc4c63)n2)cc1 ; c1ccc(-n2c3ccccc3c3c4c(ccc32)oc2c3c5ccccc5c5ccccc5c3c3ccccc3c24)nc1 ; c1ccc(-c2cc(-c3ccccc3)nc(-n3c4ccccc4c4c5c(ccc43)oc3c4c6ccccc6c6ccccc6c4c4ccccc4c35)n2)cc1]

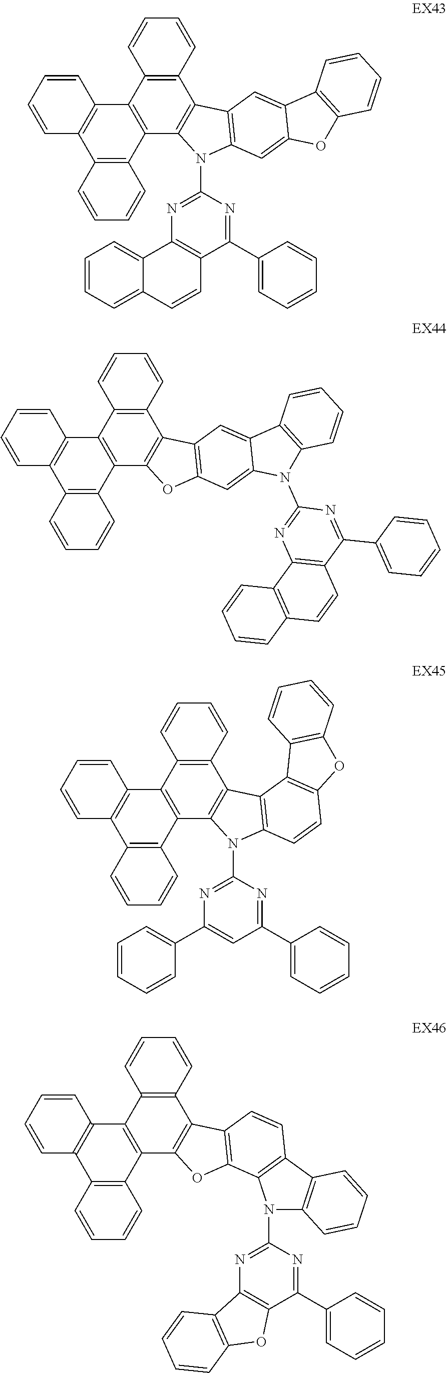

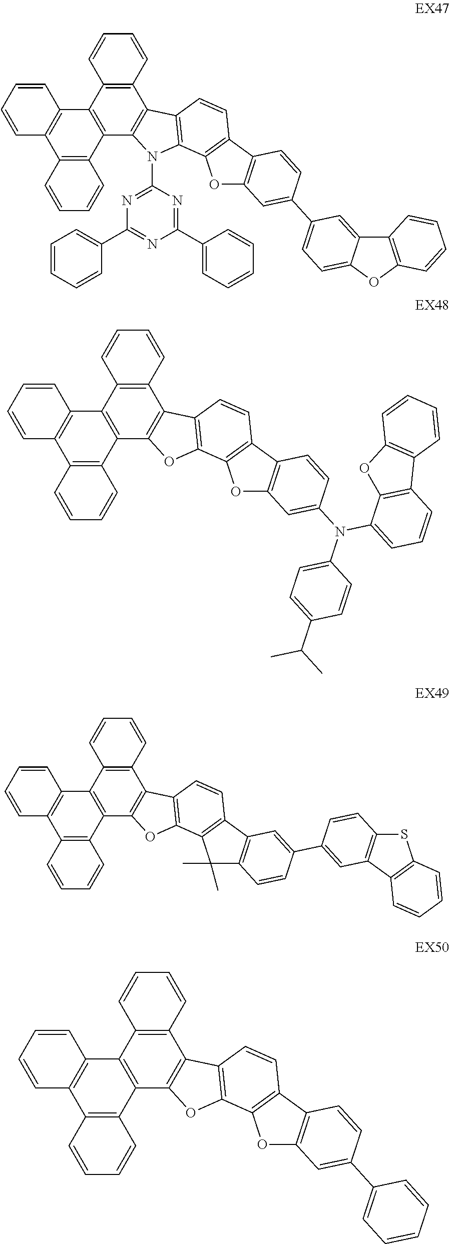 [SMILES: c1ccc(-c2nc(-c3ccccc3)nc(-n3c4c(ccc5c6ccc(-c7ccc8oc9ccccc9c8c7)cc6oc54)c4c5ccccc5c5c6ccccc6c6ccccc6c5c43)n2)cc1 ; CC(C)c1ccc(N(c2ccc3c(c2)oc2c3ccc3c2oc2c3c3ccccc3c3c4ccccc4c4ccccc4c23)c2cccc3c2oc2ccccc23)cc1 ; CC1(C)c2ccc(-c3ccc4sc5ccccc5c4c3)cc2-c2ccc3c(oc4c3c3ccccc3c3c5ccccc5c5ccccc5c43)c21 ; c1ccc(-c2ccc3c(c2)oc2c3ccc3c2oc2c3c3ccccc3c3c4ccccc4c4ccccc4c23)cc1]

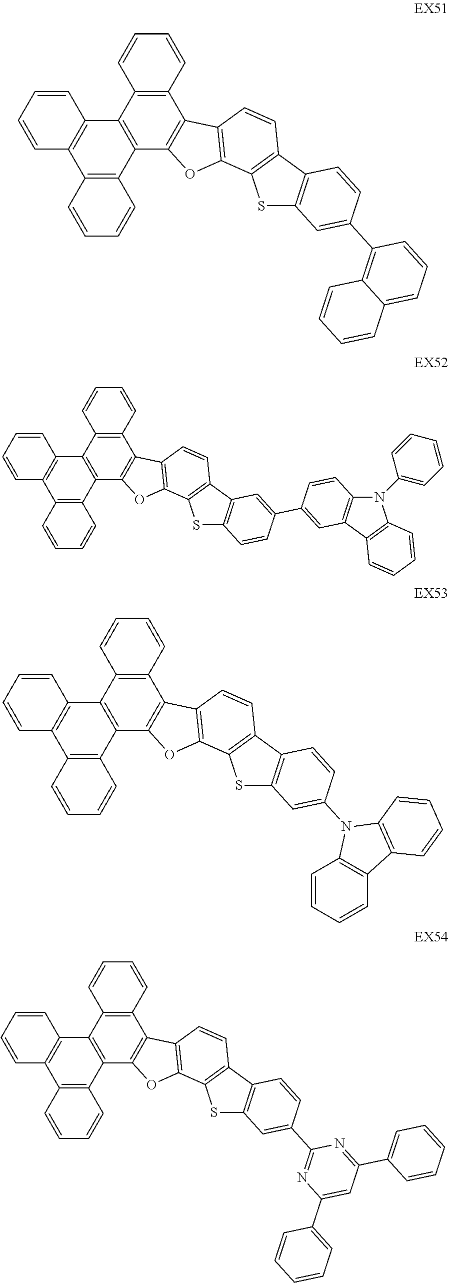 [SMILES: c1ccc2c(-c3ccc4c(c3)sc3c4ccc4c3oc3c4c4ccccc4c4c5ccccc5c5ccccc5c34)cccc2c1 ; c1ccc(-n2c3ccccc3c3cc(-c4ccc5sc6c(ccc7c6oc6c7c7ccccc7c7c8ccccc8c8ccccc8c67)c5c4)ccc32)cc1 ; c1ccc2c(c1)c1ccccc1c1c3oc4c(ccc5c6ccc(-n7c8ccccc8c8ccccc87)cc6sc54)c3c3ccccc3c21 ; c1ccc(-c2cc(-c3ccccc3)nc(-c3ccc4c(c3)sc3c4ccc4c3oc3c4c4ccccc4c4c5ccccc5c5ccccc5c34)n2)cc1]

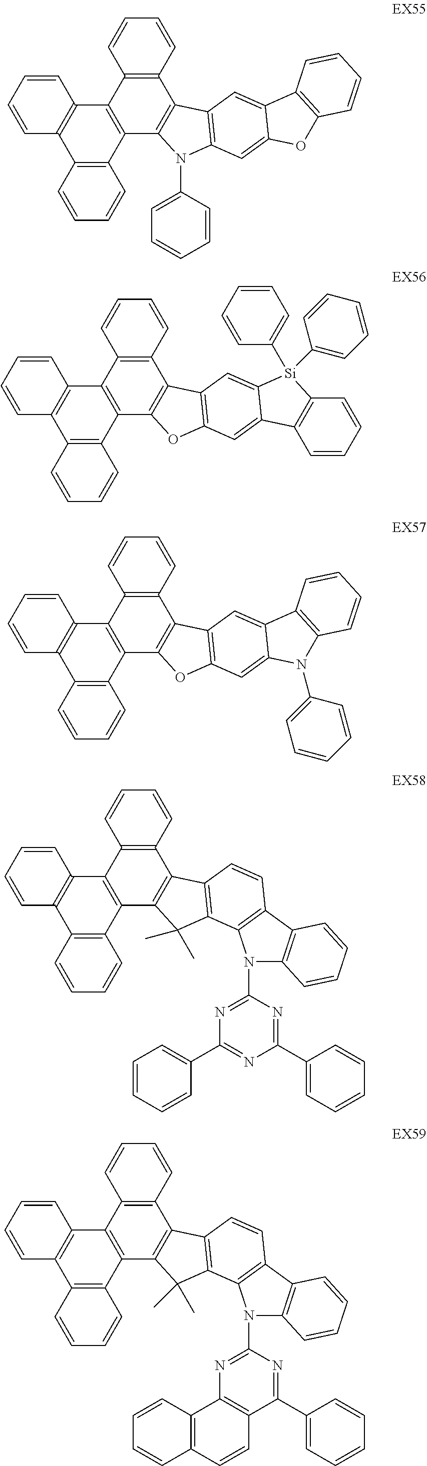

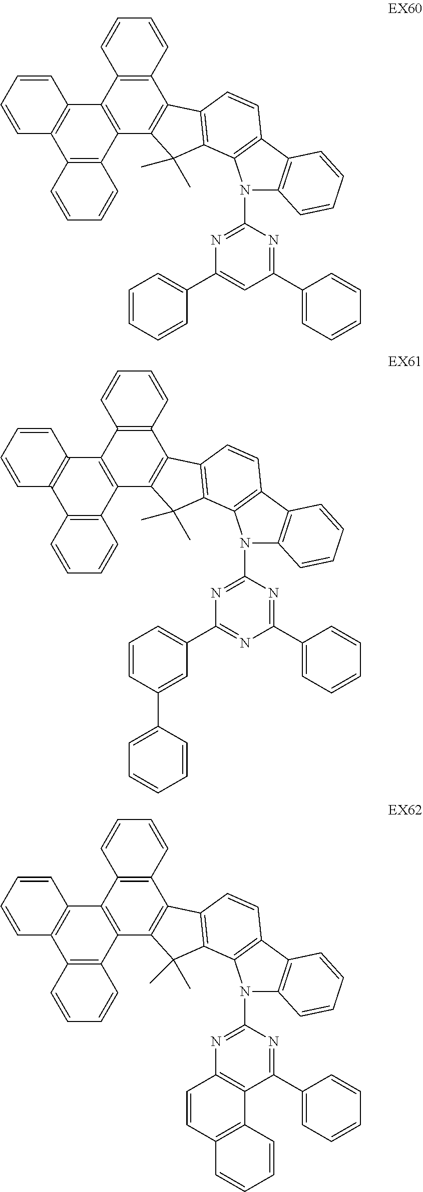

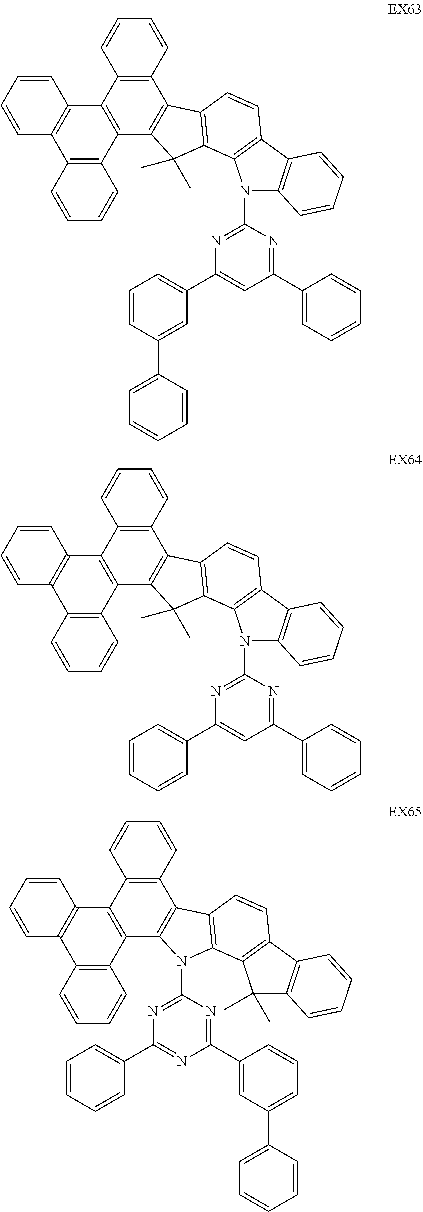

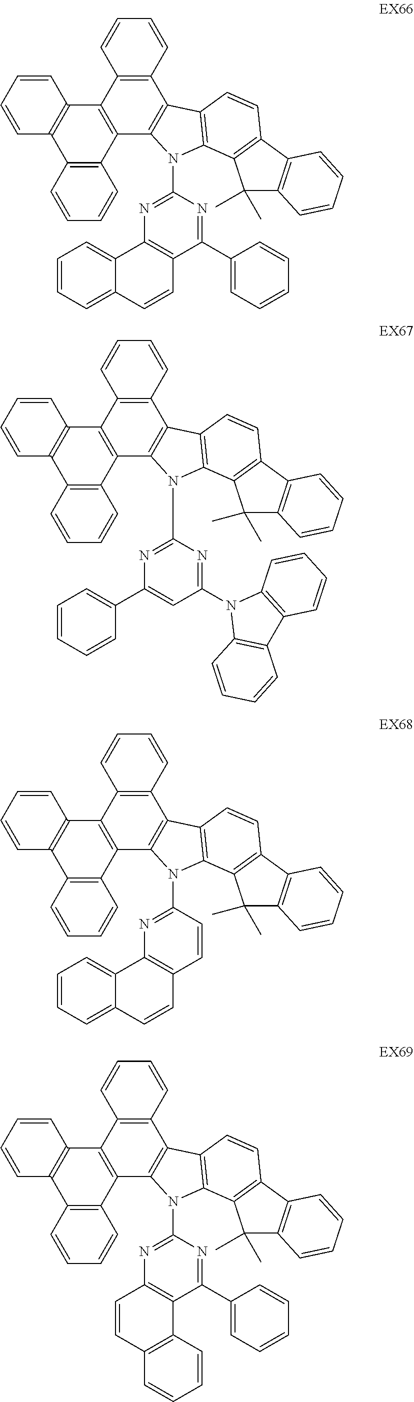

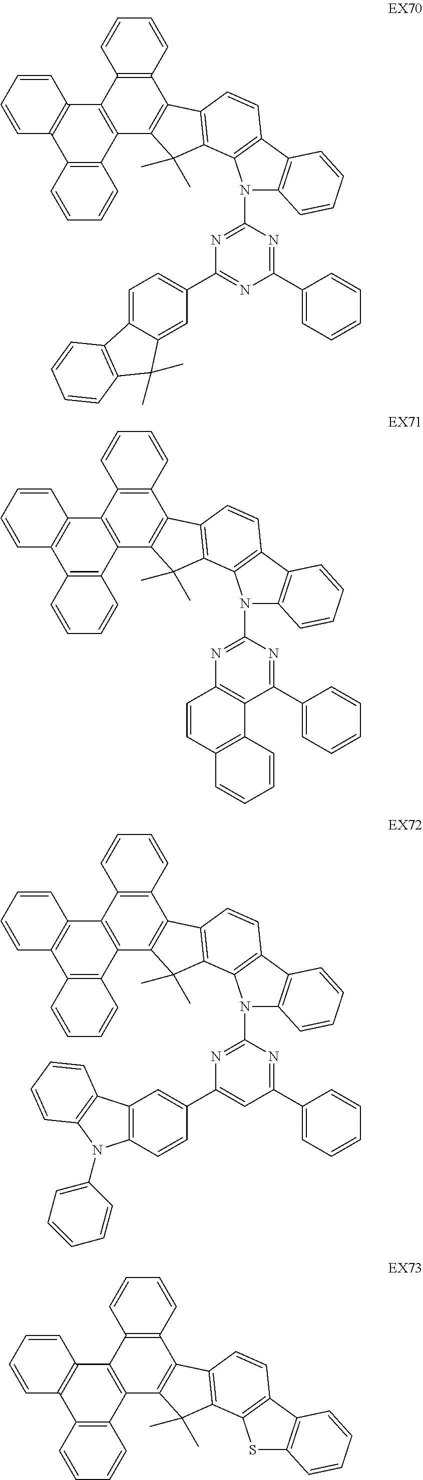

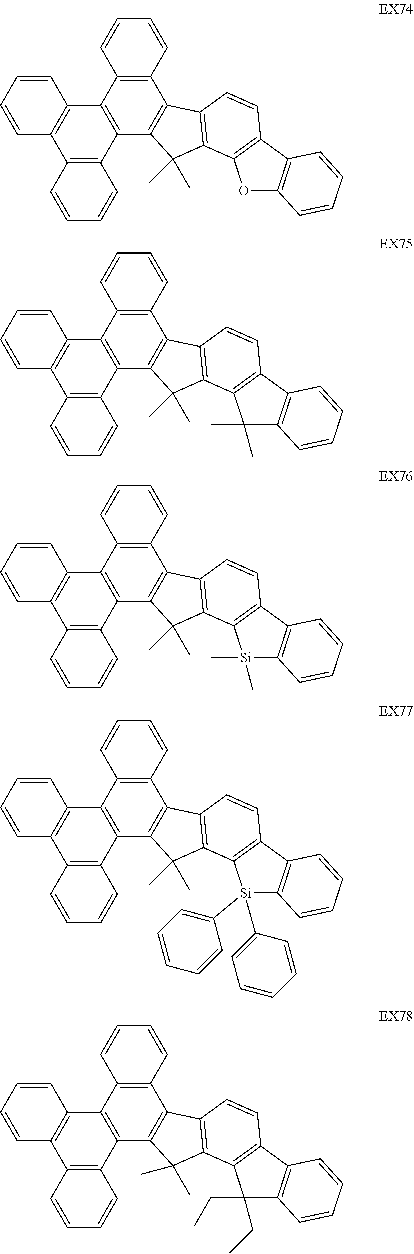

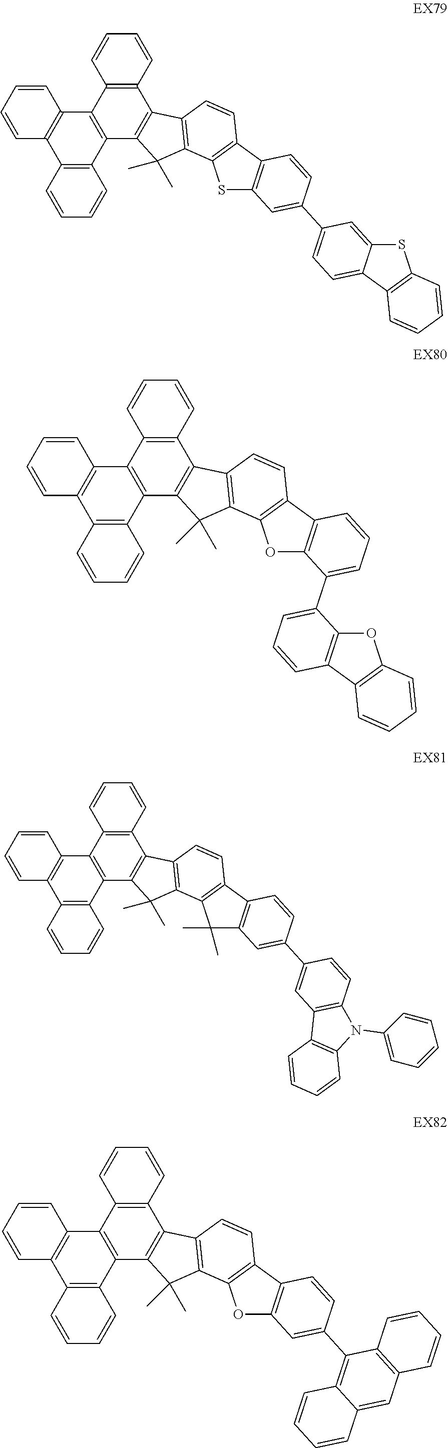 [SMILES: CC1(C)c2c(ccc3c2sc2cc(-c4ccc5c(c4)sc4ccccc45)ccc23)-c2c1c1c3ccccc3c3ccccc3c1c1ccccc21 ; CC1(C)c2c(ccc3c2oc2c(-c4cccc5c4oc4ccccc45)cccc23)-c2c1c1c3ccccc3c3ccccc3c1c1ccccc21 ; CC1(C)c2cc(-c3ccc4c(c3)c3ccccc3n4-c3ccccc3)ccc2-c2ccc3c(c21)C(C)(C)c1c-3c2ccccc2c2c3ccccc3c3ccccc3c12 ; CC1(C)c2c(ccc3c2oc2cc(-c4c5ccccc5cc5ccccc45)ccc23)-c2c1c1c3ccccc3c3ccccc3c1c1ccccc21]

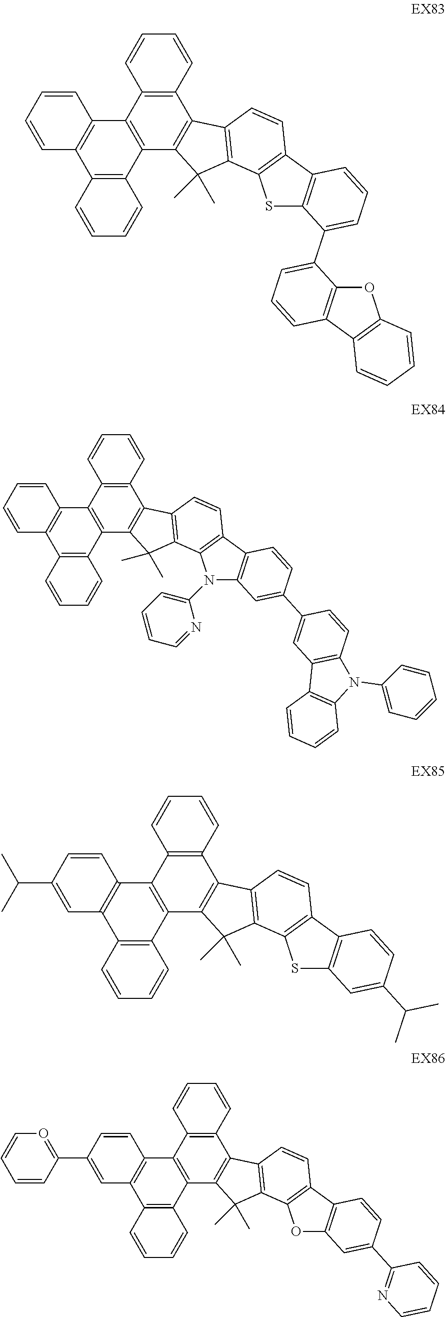

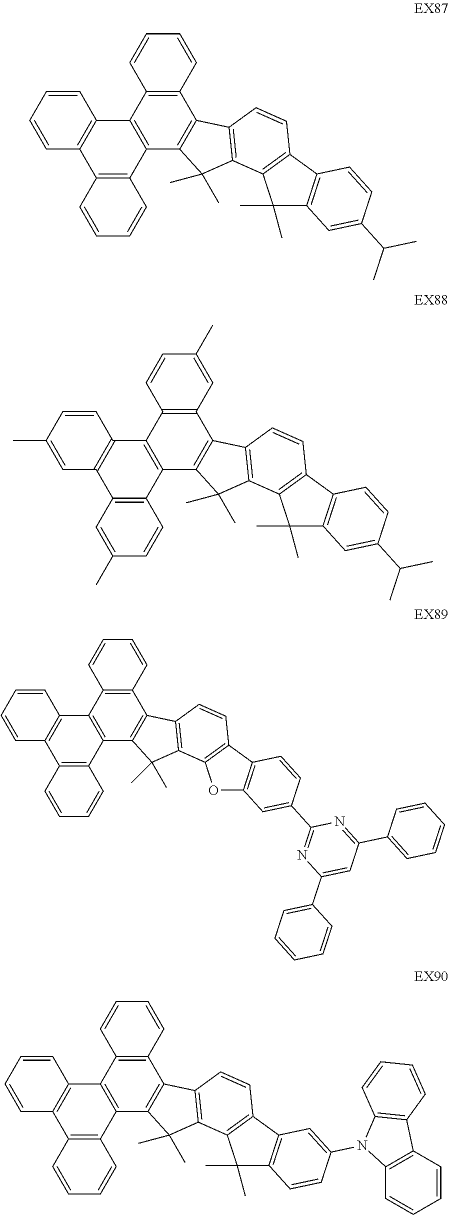

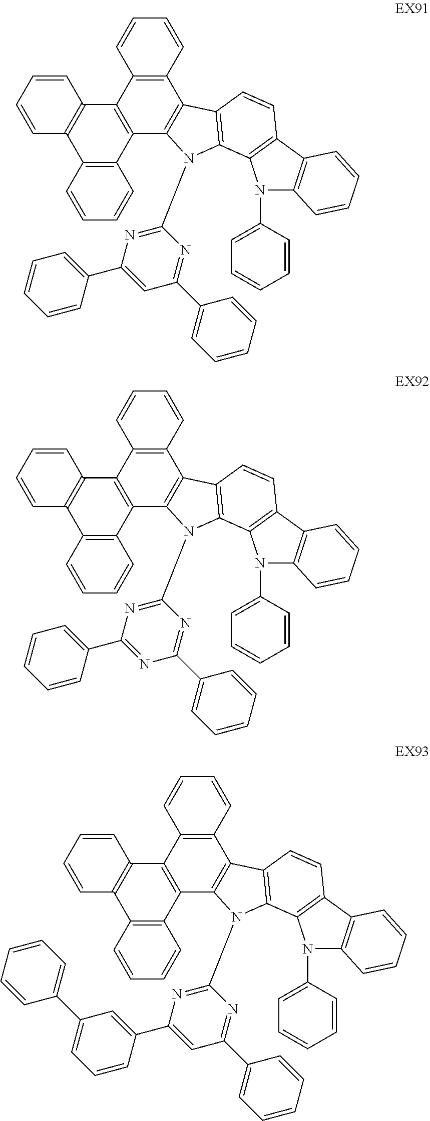

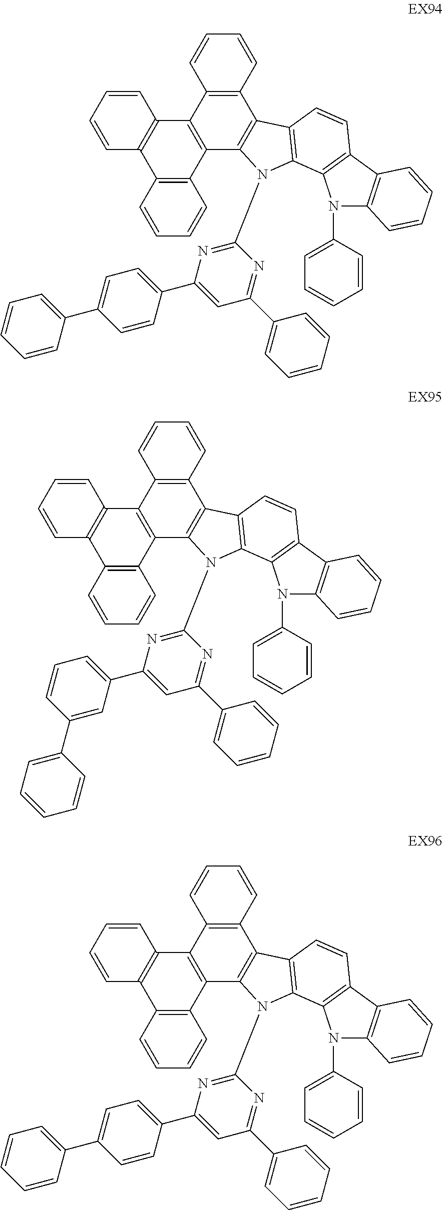 [SMILES: c1ccc(-c2ccc(-c3cc(-c4ccccc4)nc(-n4c5c(c6ccccc6c6c7ccccc7c7ccccc7c65)c5ccc6c7ccccc7n(-c7ccccc7)c6c54)n3)cc2)cc1 ; c1ccc(-c2cccc(-c3cc(-c4ccccc4)nc(-n4c5c(c6ccccc6c6c7ccccc7c7ccccc7c65)c5ccc6c7ccccc7n(-c7ccccc7)c6c54)n3)c2)cc1 ; c1ccc(-c2ccc(-c3cc(-c4ccccc4)nc(-n4c5c(c6ccccc6c6c7ccccc7c7ccccc7c65)c5ccc6c7ccccc7n(-c7ccccc7)c6c54)n3)cc2)cc1]

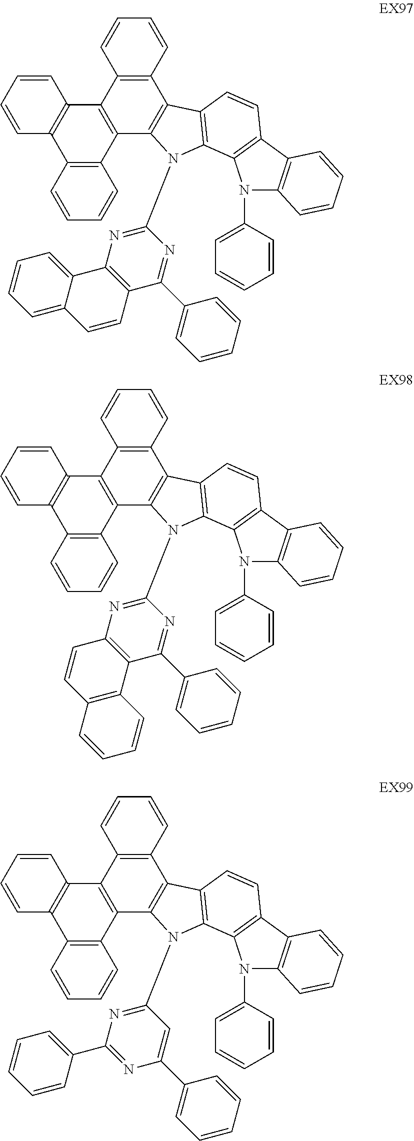

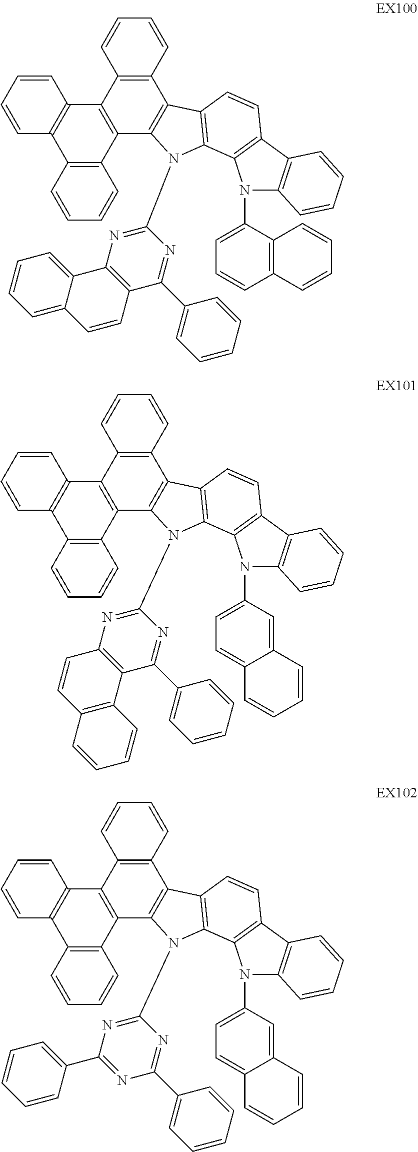

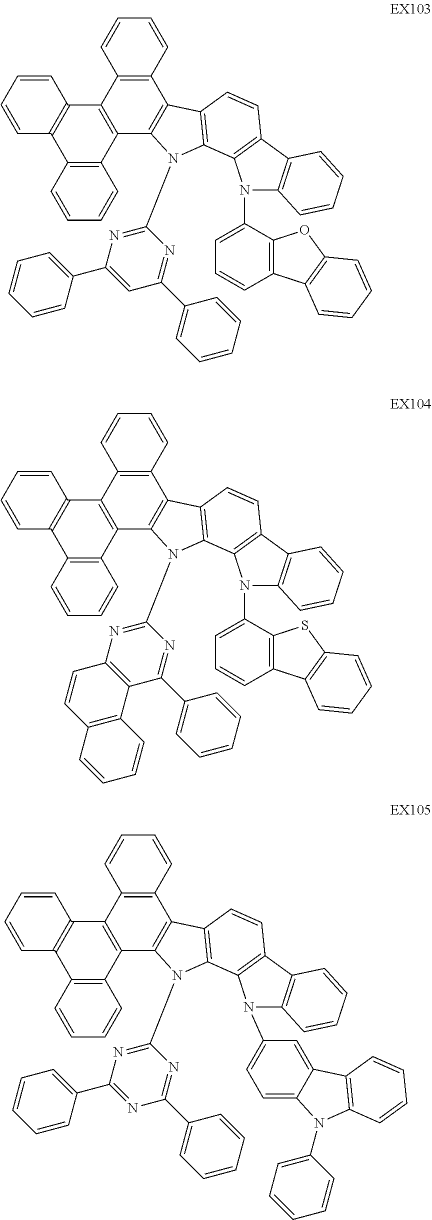 [SMILES: c1ccc(-c2cc(-c3ccccc3)nc(-n3c4c(c5ccccc5c5c6ccccc6c6ccccc6c54)c4ccc5c6ccccc6n(-c6cccc7c6oc6ccccc67)c5c43)n2)cc1 ; c1ccc(-c2nc(-n3c4c(c5ccccc5c5c6ccccc6c6ccccc6c54)c4ccc5c6ccccc6n(-c6cccc7c6sc6ccccc67)c5c43)nc3ccc4ccccc4c23)cc1 ; c1ccc(-c2nc(-c3ccccc3)nc(-n3c4c(c5ccccc5c5c6ccccc6c6ccccc6c54)c4ccc5c6ccccc6n(-c6ccc7c(c6)c6ccccc6n7-c6ccccc6)c5c43)n2)cc1]

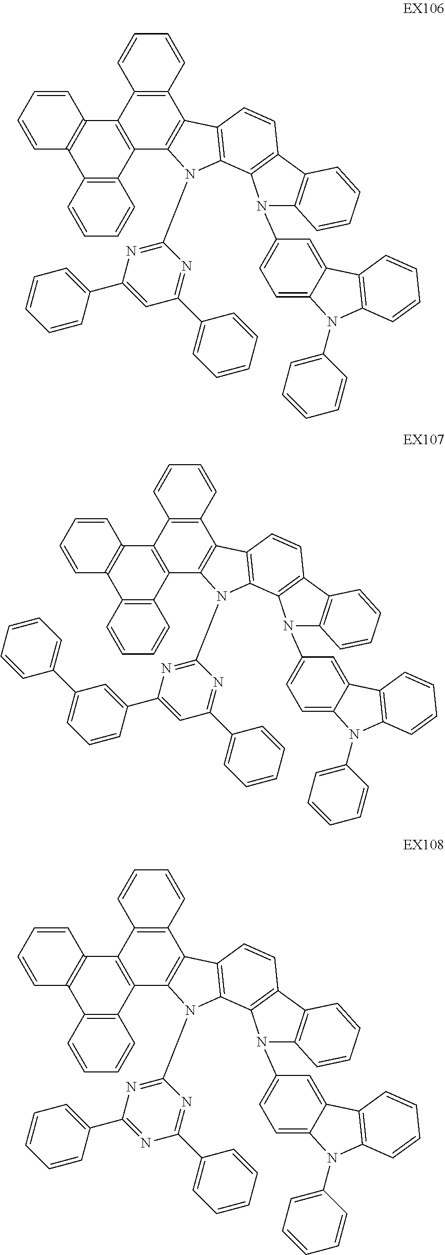 [SMILES: c1ccc(-c2cc(-c3ccccc3)nc(-n3c4c(c5ccccc5c5c6ccccc6c6ccccc6c54)c4ccc5c6ccccc6n(-c6ccc7c(c6)c6ccccc6n7-c6ccccc6)c5c43)n2)cc1 ; c1ccc(-c2cccc(-c3cc(-c4ccccc4)nc(-n4c5c(c6ccccc6c6c7ccccc7c7ccccc7c65)c5ccc6c7ccccc7n(-c7ccc8c(c7)c7ccccc7n8-c7ccccc7)c6c54)n3)c2)cc1 ; c1ccc(-c2nc(-c3ccccc3)nc(-n3c4c(c5ccccc5c5c6ccccc6c6ccccc6c54)c4ccc5c6ccccc6n(-c6ccc7c(c6)c6ccccc6n7-c6ccccc6)c5c43)n2)cc1]

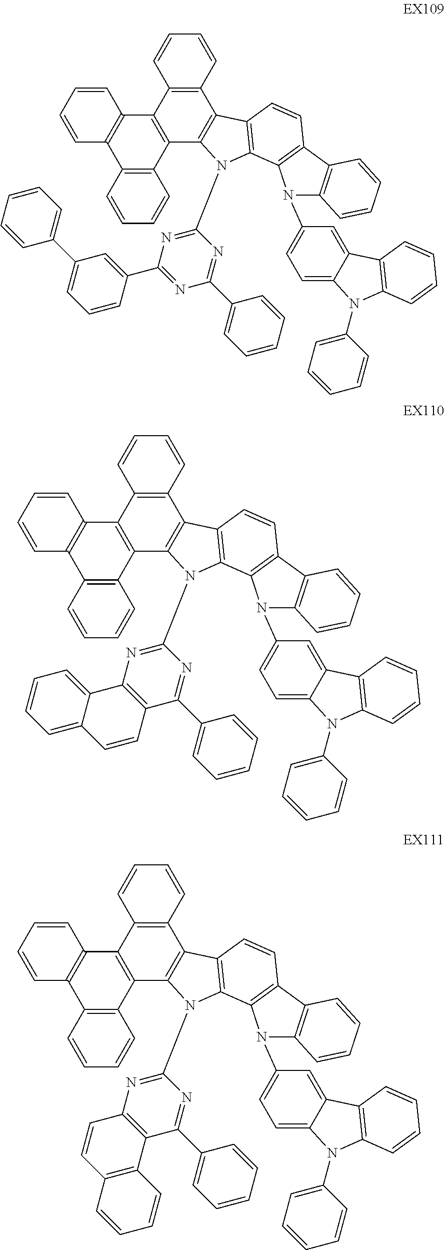 [SMILES: c1ccc(-c2cccc(-c3nc(-c4ccccc4)nc(-n4c5c(c6ccccc6c6c7ccccc7c7ccccc7c65)c5ccc6c7ccccc7n(-c7ccc8c(c7)c7ccccc7n8-c7ccccc7)c6c54)n3)c2)cc1 ; c1ccc(-c2nc(-n3c4c(c5ccccc5c5c6ccccc6c6ccccc6c54)c4ccc5c6ccccc6n(-c6ccc7c(c6)c6ccccc6n7-c6ccccc6)c5c43)nc3c2ccc2ccccc23)cc1 ; c1ccc(-c2nc(-n3c4c(c5ccccc5c5c6ccccc6c6ccccc6c54)c4ccc5c6ccccc6n(-c6ccc7c(c6)c6ccccc6n7-c6ccccc6)c5c43)nc3ccc4ccccc4c23)cc1]

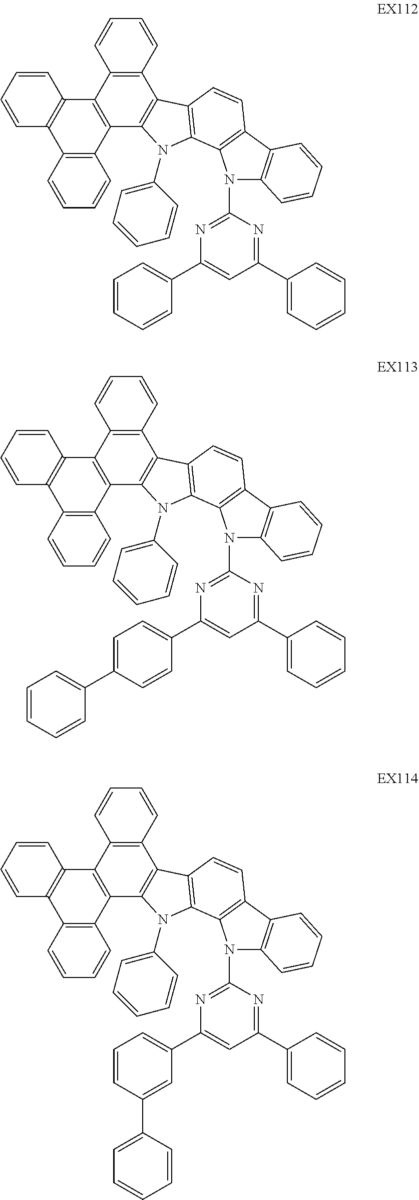 [SMILES: c1ccc(-c2cc(-c3ccccc3)nc(-n3c4ccccc4c4ccc5c6c7ccccc7c7c8ccccc8c8ccccc8c7c6n(-c6ccccc6)c5c43)n2)cc1 ; c1ccc(-c2ccc(-c3cc(-c4ccccc4)nc(-n4c5ccccc5c5ccc6c7c8ccccc8c8c9ccccc9c9ccccc9c8c7n(-c7ccccc7)c6c54)n3)cc2)cc1 ; c1ccc(-c2cccc(-c3cc(-c4ccccc4)nc(-n4c5ccccc5c5ccc6c7c8ccccc8c8c9ccccc9c9ccccc9c8c7n(-c7ccccc7)c6c54)n3)c2)cc1]

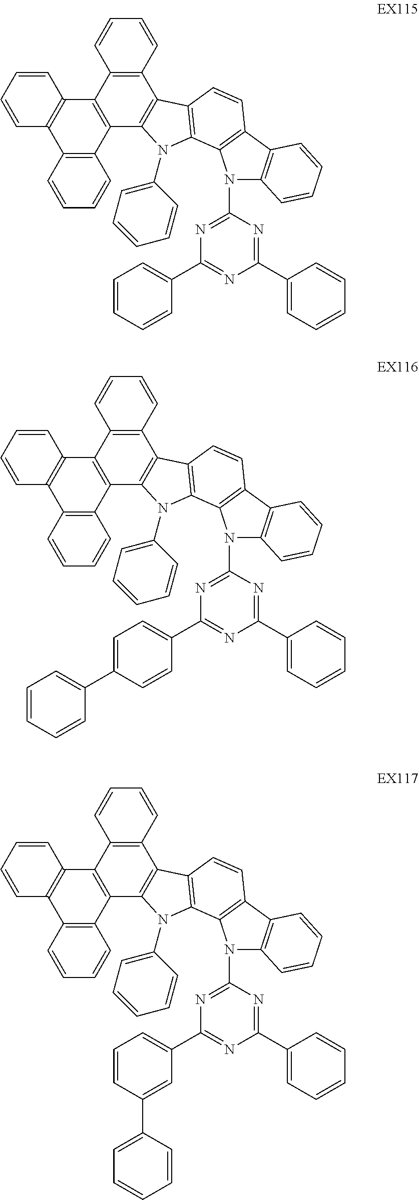 [SMILES: c1ccc(-c2nc(-c3ccccc3)nc(-n3c4ccccc4c4ccc5c6c7ccccc7c7c8ccccc8c8ccccc8c7c6n(-c6ccccc6)c5c43)n2)cc1 ; c1ccc(-c2ccc(-c3nc(-c4ccccc4)nc(-n4c5ccccc5c5ccc6c7c8ccccc8c8c9ccccc9c9ccccc9c8c7n(-c7ccccc7)c6c54)n3)cc2)cc1 ; c1ccc(-c2cccc(-c3nc(-c4ccccc4)nc(-n4c5ccccc5c5ccc6c7c8ccccc8c8c9ccccc9c9ccccc9c8c7n(-c7ccccc7)c6c54)n3)c2)cc1]

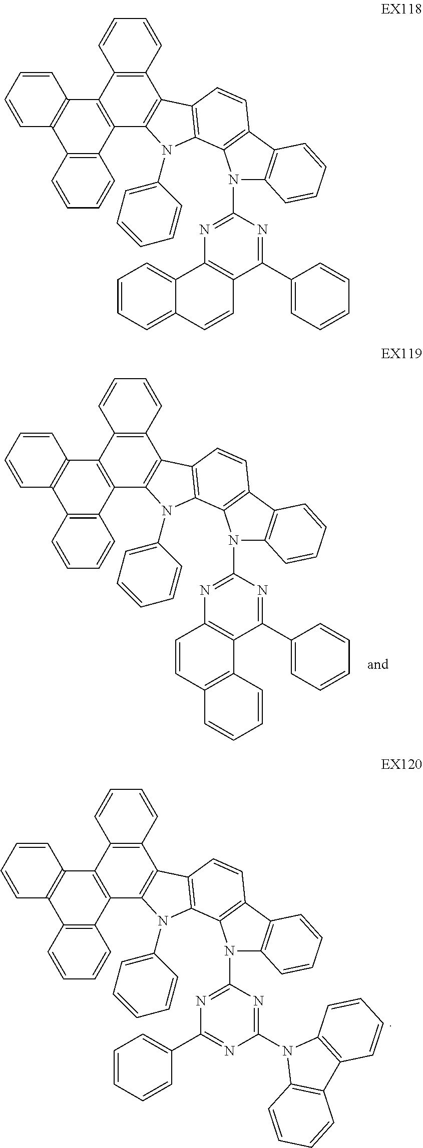 [SMILES: c1ccc(-c2nc(-n3c4ccccc4c4ccc5c6c7ccccc7c7c8ccccc8c8ccccc8c7c6n(-c6ccccc6)c5c43)nc3c2ccc2ccccc23)cc1 ; c1ccc(-c2nc(-n3c4ccccc4c4ccc5c6c7ccccc7c7c8ccccc8c8ccccc8c7c6n(-c6ccccc6)c5c43)nc3ccc4ccccc4c23)cc1 ; c1ccc(-c2nc(-n3c4ccccc4c4ccccc43)nc(-n3c4ccccc4c4ccc5c6c7ccccc7c7c8ccccc8c8ccccc8c7c6n(-c6ccccc6)c5c43)n2)cc1]

7. An organic electroluminescence device comprising an anode, a cathode and one or more organic layers formed between the anode and the cathode, wherein at least one of the organic layers comprises the organic compound according to claim 1.

8. The organic electroluminescence device according to claim 7, wherein the organic layers comprise an emissive layer having a host, and wherein the organic compound is comprised as the host.

9. The organic electroluminescence device according to claim 8, wherein the host is a fluorescent host.

10. The organic electroluminescence device according to claim 8, wherein the host is a phosphorescent host.

11. The organic electroluminescence device according to claim 7, wherein the organic layers comprise an electron transport layer, and wherein the the organic compound of claim 1 is comprised as the electron transport layer.

12. The organic electroluminescence device according to claim 7, wherein the organic layers comprise a hole blocking layer, and wherein the organic compound of claim 1 is comprised as the hole blocking layer.

13. The organic electroluminescent device according to claim 7, wherein the organic electroluminescence device is a lighting panel.

14. The organic electroluminescent device according to claim 7, wherein the organic electroluminescence device is a backlight panel.

15. An organic compound represented by the following formula (I): 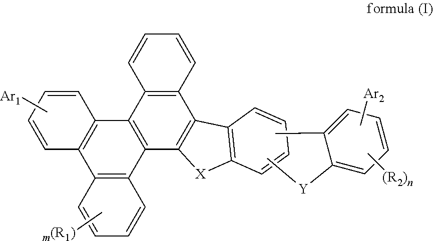
wherein X and Y are independently O, S, NR, C(CH₃)₂, C(CH₂CH₃)₂, Si(CH₃)₂ or Si(CH₂CH₃)₂;
wherein R is selected from the group consisting of aryl, aza-aryl, and combinations thereof;
wherein R is optionally substituted with phenyl, biphenyl, tricyclic aryl or tricyclic aza-aryl;
wherein Ar₁ and Ar₂ are independently absent or selected from the group consisting of methyl, isopropyl, isobutyl, phenyl, pyridinyl, and combinations thereof;
wherein m is an integer of 0, 1, 2, 3, 4, 5, 6, 7, 8, 9, 10 or 11; and
wherein n is an integer of 0, 1, 2 or 3.

16. An organic compound represented by one of the following formula (1) to formula (10): 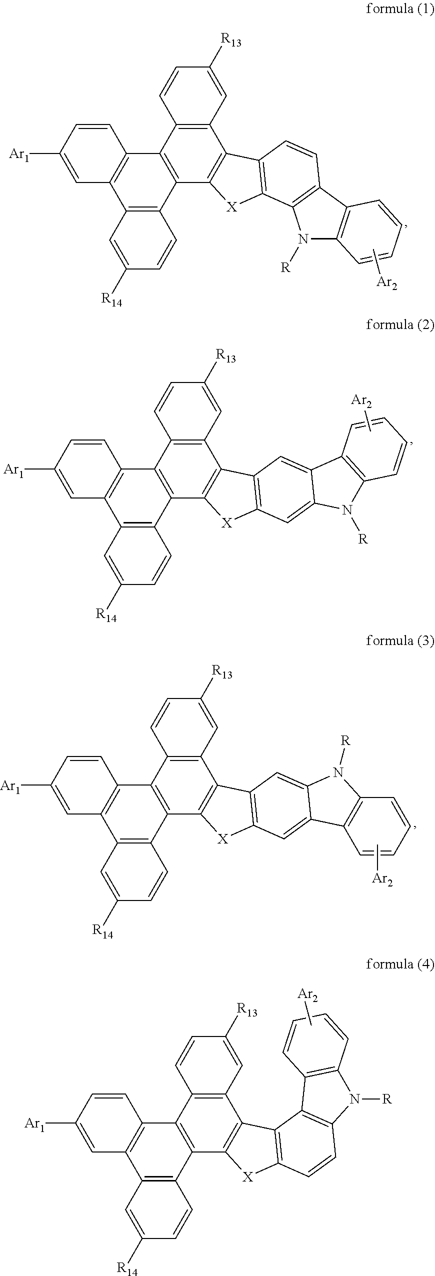 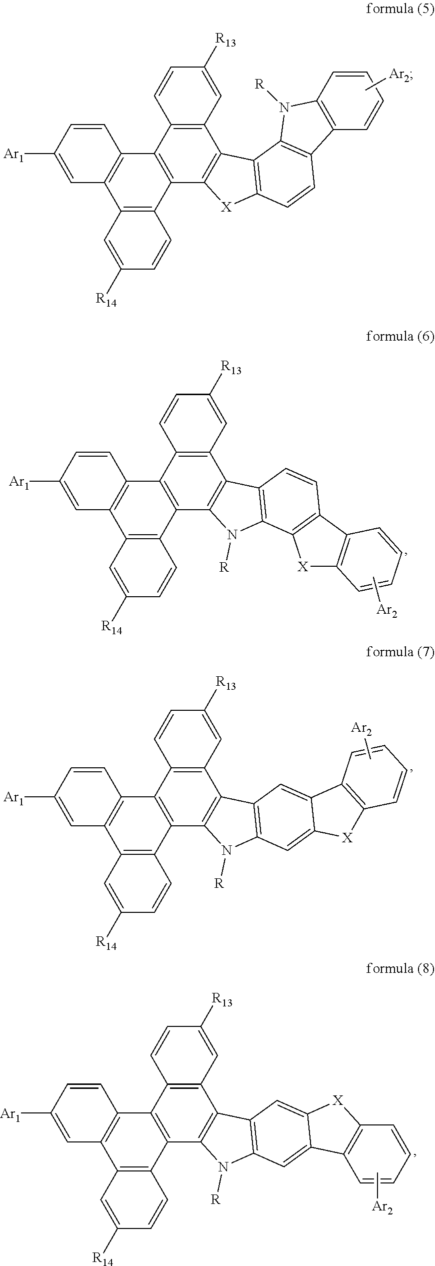 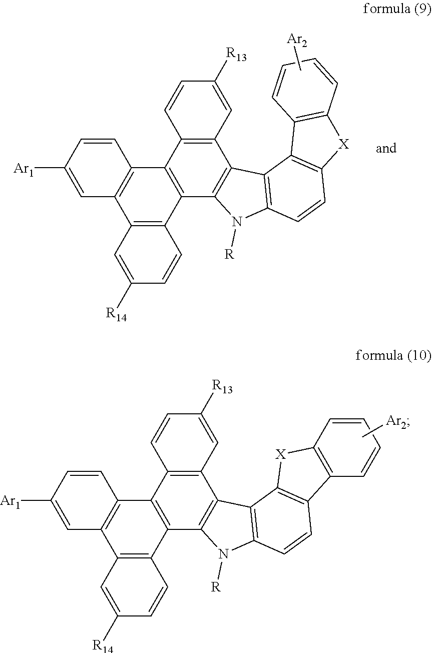
wherein Ar₁ and Ar₂ are independently absent or selected from the group consisting of methyl, isopropyl, isobutyl, phenyl, pyridinyl, and combinations thereof;
wherein X is O, S, C(CH₃)₂ or C(CH₂CH₃)2;
wherein R is selected from the group consisting of aryl, aza-aryl, and combinations thereof;
wherein R is optionally substituted with phenyl, biphenyl, tricyclic aryl or tricyclic aza-aryl; and
wherein R₁₃ and R₁₄ are independently absent or methyl.

17. The organic compound of claim 16, wherein R is selected from the group consisting of monocyclic aryl, polycyclic aryl, monocyclic aza-aryl, polycyclic aza-aryl, and combinations thereof;

18. The organic compound of claim 16, wherein R is selected from the group consisting of monocyclic aryl, tricyclic aryl, monocyclic aza-aryl, tricyclic aza-aryl, and combinations thereof.

19. The organic compound of claim 18, wherein tricyclic aza-aryl is selected from the group consisting of the following substituents:

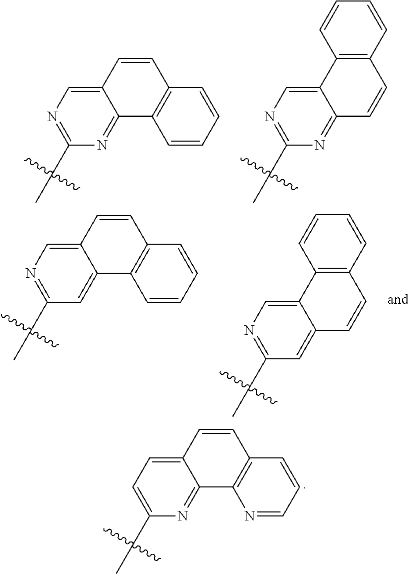

20. The organic compound of claim 18, wherein monocyclic aza-aryl is selected from the group consisting of the following substituents: 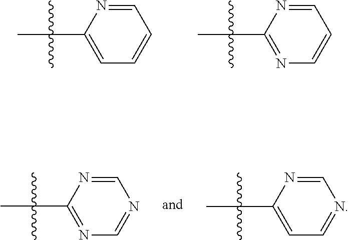 [SMILES: *c1ccccn1 ; *c1ncccn1 ; *c1ncncn1 ; *c1ccncn1]
